SEQUENCE LISTING (26 sequences)

SEQ ID NO: 1 - DNA, 890 bp, Homo sapiens
ctggccacaccctccgcctggacgcagcagccaccgccgcgtccctctctccacgaggctgccggcttaggacccccagctccgacatgtcgccctctggtcgcctgtgtcttctcaccatcgttggcctgattctccccaccagaggacagacgttgaaagataccacgtccagttcttcagcagactcaactatcatggacattcaggtcccgacacgagccccagatgcagtctacacagaactccagcccacctctccaaccccaacctggcctgctgatgaaacaccacaaccccagacccagacccagcaactggaaggaacggatgggcctctagtgacagatccagagacacacaagagcaccaaagcagctcatcccactgatgacaccacgacgctctctgagagaccatccccaagcacagacgtccagacagacccccagaccctcaagccatctggttttcatgaggatgaccccttcttctatgatgaacacaccctccggaaacgggggctgttggtcgcagctgtgctgttcatcacaggcatcatcatcctcaccagtggcaagtgcaggcagctgtcccggttatgccggaatcgttgcaggtgagtccatcagaaacaggagctgacaacctgctgggcacccgaagaccaagccccctgccagctcaccgtgcccagcctcctgcatcccctcgaagagcctggccagagagggaagacacagatgatgaagctggagccagggctgccggtccgagtctcctacctcccccaaccctgcccgcccctgaaggctacctggcgccttgggggctgtccctcaagttatctcctctgctaagacaaaaagtaaagcactgtggtctttgcc

SEQ ID NO: 2 - protein, 178 aa, Homo sapiens
MSPSGRLCLLTIVGLILPTRGQTLKDTTSSSSADSTIMDIQVPTRAPDAVYTELQPTSPTPTWPADETPQPQTQTQQLEGTDGPLVTDPETHKSTKAAHPTDDTTTLSERPSPSTDVQTDPQTLKPSGFHEDDPFFYDEHTLRKRGLLVAAVLFITGIIILTSGKCRQLSRLCRNRCR

SEQ ID NO: 3 - protein, 15 aa, Homo sapiens
TRGQTLKDTTSSSSA

SEQ ID NO: 4 - protein, 29 aa, Homo sapiens
LQPTSPTPTWPADETPQPQTQTQQLEGTD

SEQ ID NO: 5 - protein, 9 aa, Homo sapiens
PETHKSTKA

SEQ ID NO: 6 - protein, 26 aa, Homo sapiens
PTDDTTTLSERPSPSTDVQTDPQTLK

SEQ ID NO: 7 - protein, 9 aa, Homo sapiens
SGFHEDDPF

SEQ ID NO: 8 - DNA, 537 bp, Homo sapiens
atgtcgccctctggtcgcctgtgtcttctcaccatcgttggcctgattctccccaccagaggacagacgttgaaagataccacgtccagttcttcagcagactcaactatcatggacattcaggtcccgacacgagccccagatgcagtctacacagaactccagcccacctctccaaccccaacctggcctgctgatgaaacaccacaaccccagacccagacccagcaactggaaggaacggatgggcctctagtgacagatccagagacacacaagagcaccaaagcagctcatcccactgatgacaccacgacgctctctgagagaccatccccaagcacagacgtccagacagacccccagaccctcaagccatctggttttcatgaggatgaccccttcttctatgatgaacacaccctccggaaacgggggctgttggtcgcagctgtgctgttcatcacaggcatcatcatcctcaccagtggcaagtgcaggcagctgtcccggttatgccggaatcgttgcaggtga

SEQ ID NO: 9 - DNA, 1631 bp, Homo sapiens
ctggccacaccctccgcctggacgcagcagccaccgccgcgtccctctctccacgaggctgccggcttaggacccccagctccgacgtaagtccctctcgcgcgccacctccatccgctgcccctctgcccacgggccgggctcaggtgggtaggggtttgaccccttccgctcctctccgccccccatccacgcccacatctctctgcctcctctcctgggctacagtgggccgctctgcctcgctcccgcccgcctgcctcagtctccccagctctctgccacccccctcggaccagtgcctcttaggtctctccagcgcgccatccctgcaaggcggcccctgacaccaactcactttttcctctgagcgcgggaggcgttactcactcttcctccttcccctcctcgccgactcccgggctctgggcccctgggggagggaagaggcctcctatctactcctctgccccagtcccctccttgcgtcagtcccagtgagggataagcgcctggcggaaggcgcagggaggtgtttctctgcttcaggagtgcccgccggcccttgcagctgctggaagacccatttatctcatgcttcttgttttctttggggacctgcaggggaaggaagcagggtgacggtttggtatccccacctaagaccctcccctttcccctgaggccagccgtcagcccctggcagggggtcttggaagccagaggtttttgctcagggcagggaaagggctgcaggatccccgggggctgccggaggtcggtctcactgacatcatggctgaccccagcatcgcctggtcccacagatgtcgccctctggtcgcctgtgtcttctcaccatcgttggcctgattctccccaccagaggacagacgttgaaagataccacgtccagttcttcagcagactcaactatcatggacattcaggtcccgacacgagccccagatgcagtctacacagaactccagcccacctctccaaccccaacctggcctgctgatgaaacaccacaaccccagacccagacccagcaactggaaggaacggatgggcctctagtgacagatccagagacacacaagagcaccaaagcagctcatcccactgatgacaccacgacgctctctgagagaccatccccaagcacagacgtccagacagacccccagaccctcaagccatctggttttcatgaggatgaccccttcttctatgatgaacacaccctccggaaacgggggctgttggtcgcagctgtgctgttcatcacaggcatcatcatcctcaccagtggcaagtgcaggcagctgtcccggttatgccggaatcgttgcaggtgagtccatcagaaacaggagctgacaacctgctgggcacccgaagaccaagccccctgccagctcaccgtgcccagcctcctgcatcccctcgaagagcctggccagagagggaagacacagatgatgaagctggagccagggctgccggtccgagtctcctacctcccccaaccctgcccgcccctgaaggctacctggcgccttgggggctgtccctcaagttatctcctctgctaagacaaaaagtaaagcactgtggtctttgcc

SEQ ID NO: 10 - protein, 145 aa, Homo sapiens
MSPSGRLCLLTIVGLILPTRGQTLKDTTSSSSADSTIMDIQVPTRAPDAVYTELQPTSPTPTWPADETPQPQTQTQQLEGTDGPLVTDPETHKSTKAAHPTDDTTTLSERPSPSTDVQTDPQTLKPSGFHEDDPFFYDEHTLRKR

SEQ ID NO: 11 - protein, 21 aa, Homo sapiens
MSPSGRLCLLTIVGLILPTRG

SEQ ID NO: 12 - DNA, 24 bp, Artificial Sequence
  Primer
ggtgagaagacacaggcgaccaga

SEQ ID NO: 13 - DNA, 25 bp, Artificial Sequence
  Primer
ttgagtctgctgaagaactggacgt

SEQ ID NO: 14 - DNA, 25 bp, Artificial Sequence
  Primer
gtgtcgggacctgaatgtccatgat

SEQ ID NO: 15 - DNA, 25 bp, Artificial Sequence
  Primer
gagaggtgggctggagttctgtgta

SEQ ID NO: 16 - DNA, 24 bp, Artificial Sequence
  Primer
atccgttccttccagttgctgggt

SEQ ID NO: 17 - DNA, 24 bp, Artificial Sequence
  Primer
gcgtcgtggtgtcatcagtgggat

SEQ ID NO: 18 - DNA, 25 bp, Artificial Sequence
  Primer
ggatgatgatgcctgtgatgaacag

SEQ ID NO: 19 - DNA, 25 bp, Artificial Sequence
  Primer
tgatggactcacctgcaacgattcc

SEQ ID NO: 20 - DNA, 21 bp, Artificial Sequence
  Primer
cccggttatgccggaatcgtt

SEQ ID NO: 21 - DNA, 21 bp, Artificial Sequence
  Primer
ctcaactatcatggacattca

SEQ ID NO: 22 - DNA, 21 bp, Artificial Sequence
  Primer
gaggacagacgttgaaagata

SEQ ID NO: 23 - DNA, 21 bp, Artificial Sequence
  Primer
ccagatgcagtctacacagaa

SEQ ID NO: 24 - DNA, 21 bp, Artificial Sequence
  Primer
caggcatcatcatcctcacca

SEQ ID NO: 25 - DNA, 21 bp, Artificial Sequence
  Primer
caggtgagtccatcagaaaca

SEQ ID NO: 26 - DNA, 21 bp, Artificial Sequence
  Primer
gagggaagacacagatgatga

(19) United States
(12) Patent Application Publication
(10) Pub. No.: US 2010/0154071 A1
(43) Pub. Date: Jun. 17, 2010

(54) FXDY5 MODULATORS FOR TREATING, DIAGNOSING, AND DETECTING CANCER

(75) Inventors: Abdallah Fanidi, Emeryville, CA (US); Mary Jo Janatpour, Emeryville, CA (US); Robert Q. To, Fremont, CA (US); Deborah Zimmerman, Oakland, CA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/450,132

(22) PCT Filed: Mar. 28, 2008
(86) PCT No.: PCT/US2008/058616

§ 371 Date: Nov. 13, 2009

Related U.S. Application Data
(60) Provisional application No. 60/908,580, filed on Mar. 28, 2007.

(51) Int. Cl. A01K 67/00; A61P 35/00; C12N 5/00; A61K 31/519; A61K 31/704; A61K 39/395; A61K 31/713; A61K 31/7088; A61K 49/00; C12Q 1/02; C12N 5/10; C12N 5/12; C07K 14/435; C07H 21/00; C07K 16/18

(52) U.S. Cl. 800/13; 435/375; 514/249; 514/34; 424/130.1; 514/44 R; 424/141.1; 424/139.1; 424/9.1; 435/29; 514/44 A; 435/325; 530/350; 536/23.5; 530/387.1

(57) ABSTRACT

The invention provides, inter alia, methods for treating cancer, compositions for treating cancer, and methods and compositions for diagnosing and/or detecting cancer. In particular, the present invention provides compositions and methods for treating, diagnosing and detecting cancers associated with FXYD5 overexpression.

### FIELD OF THE INVENTION

The present invention relates generally to the field of oncology. More particularly, the invention relates to methods for treating cancer, compositions for treating cancer, and methods and compositions for diagnosing and/or detecting cancer.

### BACKGROUND OF THE INVENTION

Cancer is the second leading cause of death in the United States. Although “cancer” is used to describe many different types of cancer, i.e. breast, prostate, lung, colon, pancreas, each type of cancer differs both at the phenotypic level and the genetic level. The unregulated growth characteristic of cancer occurs when the expression of one or more genes becomes dysregulated due to mutations, and cell growth can no longer be controlled.

Cancer metastasis requires changes in the expression of molecules that control cell-cell adhesion. The cadherins are a family of transmembrane glycoproteins which mediate cell-cell adhesion and the disregulation of which has been correlated with metastasis. For example, loss of expression of a primary adhesion molecule of epithelial cells, E-cildherin, has been linked to cellular transition to an invasive phenotype (Peri et al. (1998) Nature 392, 190-193). Molecules which regulate the expression or activity of E-cadherin have also been implicated in cancer metastasis. Examples of these molecules include catenina, which link E-cadherin to the cytoskeleton, and transcriptional repressors of E-cadherin expression, such as Snail and Sip-1 (Bailie et at, (2000) Nat. Cell. Biol. 2, 84-89; Comijn et al., (2001) 7. 1267-1278).

FXYD Domain-containing Ion Transport Regulator 5 (FXYD5), also known as Dysadherin, has been proposed to regulate E-cadherin expression (bo et al., (2002) Proc. Natl. Acad. Sci. USA 99(1), 365-370). FXYD5 is a member of a family of small type I membrane protein which possess a conserved 35 amino-acid core sequence. FXYD family proteins are expressed early in fetal development and are usually associated with tissues involved in fluid and solute transport (e.g., kidney, colon, breast/mammary gland, pancreas, prostate, liver, lung and placenta) as well as tissues that are electrically excitable (e.g., nervous system, muscle). FXYD family proteins are thought to be involved in the control of ion transport. Several FXYD proteins have been shown to interact with Na,K ATP-ases and modulate pump activity. FXYD5 possesses a long extracellular domain and short intracellular domain relative to other FXYD family proteins.

To date, however, the role of FXYD5 in cancer has not been fully elucidated. There is a need to identify compositions and methods that modulate FXYD5. The present invention is directed to these, as well as other, important needs.

### SUMMARY OF THE INVENTION

In some aspects, the present invention provides compositions comprising a FXYD5 modulator and one or more pharmaceutically acceptable carriers. In some embodiments the FXYD5 modulator is an isolated double-stranded RNA (dsRNA). In some embodiments the FXYD5 modulator is an isolated oligonucleotide comprising at least 10 consecutive nucleotides of a sequence of SEQ ID NO:1, or of a sequence at least 80%, 85%, 90%, 95%, 96%, 97%, 98%, or 99% identical to SEQ ID NO: I. In some embodiments the FXYD5 modulator is an antibody that binds an epitope in the extracellular domain of FXYD5. In some embodiments the FXYD5 modulator is a dsRNA, a siRNA or an antisense oligonucleotide.

In some aspects, the present invention provides methods of treating cancer or a cancer symptom in a patient in need thereof comprising administering to the patient a therapeutically effective amount of an FXYD5 modulator (e.g., an FXYD5 inhibitor).

In some aspects, the present invention provides methods of modulating a FXYD5-related biological activity in a patient comprising administering to the patient an amount of a FXYD5 modulator effective to modulate the FXYD5-related biological activity.

In some aspects, the present invention provides methods of identifying a patient susceptible to FXYD5 therapy comprising detecting the presence or absence of FXYD5 differential expression in a patient sample, administering a therapeutically effective amount of a FXYD5 modulator to the patient if the patient is a candidate for FXYD5 therapy; and administering a conventional cancer therapeutic to the patient if the patient is not a candidate for FXYD5 therapy.

In some aspects, the present invention provides methods of inhibiting growth of cancer cells that express FXYD5 comprising contacting the cells with an amount of an FXYD5 modulator effective to inhibit growth of the cells.

In some aspects, the present invention provides methods of inhibiting a cancer cell phenotype in a population of cells expressing FXYD5 comprising administering to the cell population an amount of an FXYD5 modulator (e.g., an FXYD5 inhibitor) effective to inhibit the cancer cell phenotype.

In some aspects, the present invention provides methods for detecting one or more cancer cells expressing FXYD5 in a sample comprising contacting the sample with a composition comprising an FXYD5 modulator linked to an imaging agent and detecting the localization of the imaging agent in the sample.

In some aspects, the present invention provides methods for increasing the interaction of two or more cells, at least one of which cells expresses FXYD5, comprising administering an effective amount of an FXYD5 modulator to a sample comprising the cells. In various embodiments, the F. modulator is an FXYD5 antagonist which increases the interaction of two or more cells via direct or indirect modulation of cell-cell adhesive interactions. By increasing cell-cell interactions (e.g., between neoplastic cells and other cells in the body), the modulator may be effective to lessen the propensity of the neoplastic cells to metastasize.

In some aspects, the present invention provides methods of expressing an anti-FXYD5 antibody in a CHO or myeloma celL In some embodiments the anti-FXYD5 antibody inhibits one or more FXYD5-related biological activities. In some embodiments the method comprises expressing a nucleic acid encoding the anti-FXYD5 antibody in a CHO or myeloma cell.

In some aspects, the present invention provides methods of identifying a cancer inhibitor, comprising contacting a cell expressing FXYD5 with a candidate compound and a FXYD5 ligand, and determining whether an FXYD5-related activity is inhibited. In some embodiments inhibition of the FXYD5-related activity is indicative of a cancer inhibitor.

In some aspects, the present invention provides methods of identifying a cancer inhibitor comprising contacting a cell expressing FXYD5 with a candidate compound and an FXYD5 ligand, and determining whether a downstream marker of FXYD5 is inhibited. In some embodiments inhibition of the downstream marker is indicative of a cancer inhibitor.

In some aspects, the present invention provides methods for determining the susceptibility of a patient to an FXYD5 modulator comprising detecting evidence of differential expression of FXYD5 in said patient's cancer sample. In some embodiments evidence of differential expression of FXYD5 is indicative of the patient's susceptibility to a FXYD5 modulator.

In some aspects, the present invention provides methods of purifying FXYD5 protein from a sample comprising FXYD5 protein comprising providing an affinity matrix comprising a FXYDS antibody bound to a solid support, contacting the sample with the affinity matrix to form an affinity matrix-FXYD5 protein complex; separating the affinity matrix-FXYD5 protein complex from the remainder of the sample; and releasing FXYD5 protein from the affinity matrix.

In some aspects, the present invention provides methods of delivering a cytotoxic agent or a diagnostic agent to one or more cells that express FXYD5, comprising providing the cytotoxic agent or the diagnostic agent conjugated to a FXYD5 antibody or fragment thereof and exposing the cell to the antibody-agent or fragment-agent conjugate.

In some aspects, the present invention provides methods for determining the prognosis of a cancer patient detecting the presence or absence of FXYD5 bound to the plasma membrane of a cell in a sample of the patient. In some embodiments the absence of FXYDS bound to the plasma membrane of a cell in a sample of the patient indicates a good prognosis for the patient.

These and other aspects of the present invention will be elucidated in the following detailed description of the invention.

### BRIEF DESCRIPTION OF THE DRAWINGS

FIG. 1 depicts FXYDS gene expression data generated from Affymetrix GeneChip® from colon cancer, breast cancer and prostate cancer tissues and the corresponding normal colon, breast and prostate tissues.

FIG. 2 depicts FXYD5 gene expression data in normal human tissues.

FIG. 3 depicts FXYDS gene expression data in normal human tissues.

FIG. 4 an oligonucleotide array analysis of FXYD5 mRNA expression in cancerous and normal tissues.

FIG. 5 depicts an RT-PCR analysis of FXYD5 gene expression in normal human tissues.

FIG. 6 depicts an RT-PCR analysis of FXYD5 gene expression in cell lines.

FIG. 7 depicts an RT-PCR analysis of FXYD5 gene expression in normal tissues and colon cancer.

FIG. 8 depicts an RT-PCR analysis of FXYD5 gene expression in normal tissues, and breast and colon cancer.

FIG. 9 depicts gene expression of FXYD5 family members in metastatic colon cancer.

FIG. 10 depicts a FACS analysis of cell-surface expression of FXYD5 in cancer cell lines.

FIG. 11 depicts an analysis of the effect of FXYD5 antisense oligonucleotides on HT29 cell anchorage independent growth.

FIG. 12 depicts an analysis of the effect of FXYD5 antisense oligonucleotides on HT29 cell anchorage independent growth.

FIG. 13 depicts an analysis of the effect of FXYD5 siRNA on PC3 cell anchorage independent growth.

FIG. 14 depicts an analysis of the effect of FXYD5 antisense RNA on PC3 cell anchorage independent growth.

FIG. 15 depicts a cytotoxicity analysis of FXYD5 siRNA in HCT116 cells.

FIG. 16 depicts a cytotoxicity analysis of FXYD5 siRNA in MRC9 cells.

FIG. 17 depicts a cytotoxicity analysis of FXYD5 siRNA in combination with chemotherapeutic agents in LnCaP cells.

### DETAILED DESCRIPTION

The present invention provides methods and compositions for the treatment, diagnosis and imaging of cancer, in particular for the treatment, diagnosis and imaging of FXYD5-related cancer.

The inventors of the present application have discovered, Inter alfa, that FXYD5 is over-expressed in several cancers, including colon cancer, breast cancer, prostate cancer, and ovarian cancer, and has restricted expression in normal tissues. Surprisingly, inhibition of FXYD5 induces cytotoxicity in cancer cells but not in normal cells expressing FXYD5. Inhibition of FXYD5 also inhibits the ability of cancer cells to grow in an anchorage-independent manner. Furthermore, the inventors have discovered that in some embodiments, inhibition of FXYD5 in combination with chemotherapeutic treatment of cancer cells produces an additive cytotoxic effect on the cells. These and other aspects of the present invention are provided in the present application.

Definitions

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pennsylvania: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); and Sambrook at al., Molecular Cloning: A Laboratory Manual (2nd Edition, 1989).

As used herein, the singular forms “a,” “an” and “the” include plural references unless the content clearly dictates otherwise. Thus, for example, reference to “an antibody” includes a mixture of two or more such antibodies.

As used herein, the term “about” refers to ranges spanning +/−10% of a given value or ranges spanning +1-5% of a value.

As used herein, the term “FXYD5”, refers to the protein also known as FXYD Domain-containing Ion Transport Regulator 5 and Dysadherin, as well as to the nucleic acid encoding the protein (see, for example, GenBank® Ref. No. NM_{—}014164.4, GI No. 47778936, and GenBank® Ref. No. NM 144779.1, GI No. 47778937, nucleotide sequences; GenBank® Ref. No. NP 054883.3,G1 No. 21618361, amino acid sequences). Exemplary FXYD5 sequences include SEQ ID NOS:1 and 9 (nucleotide sequences) and SEQ ID NO:2 (amino acid sequence). An exemplary coding sequence of FXYD5, corresponding to nucleotides 87-623 of SEQ II) NO:1, is set forth as SEQ ID NO:8. An exemplary FXYD5 extracellular domain amino acid sequence; corresponding to amino acids 1-145 of SEQ ID NO:2, is set forth as SEQ ID NO:10. An exemplary FXYD5 signal peptide sequence, corresponding to amino acids 1-21 of SEQ ID NO:2, is set forth as SEQ 1D NO:11.

The terms “polypeptide” and “protein”, are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some embodiments the subject is a human.

As used herein, “cancer” refers to primary or metastatic cancers. The term “cancer cells” refers to cells that are transformed. These cells can be isolated from a patient who has cancer, or be cells that are transformed in vitro to become cancerous. Cancer cells can be derived from many types of samples including any tissue or cell culture line. In some embodiments the cancer cells are hyperplasias, tumor cells, or neoplasms. In some embodiments, the cancer cells are isolated from breast cancer, skin cancer, esophageal cancer, liver cancer, pancreatic cancer, prostatic cancer, uterine cancer, cervical cancer, lung cancer, bladder cancer, ovarian cancer, multiple myeloma and melanoma. In some embodiments, the cancer cells are taken from established cell lines that are publicly available. In some embodiments, cancer cells are isolated from pre-existing patient samples or from libraries comprising cancer cells. In some embodiments, cancer cells are isolated and then implanted in a different host, e.g., in a xenograft. In some embodiments cancer cells are transplanted and used in a SCID mouse model. In some embodiments, the cancer is colon cancer. In some embodiments, the cancer is breast cancer. In some embodiments, the cancer is ovarian or prostate cancer.

As used herein, the term “transformed” refers to any alteration in the properties of a cell that is stably inherited by its progeny. In some embodiments, “transformed” refers to the change of normal cell to a cancerous cell, e.g., one that is capable of causing tumors. In some embodiments, a transformed cell is immortalized. Transformation can be caused by a number of factors, including overexpression of a receptor in the absence of receptor phosphorylation, viral infection, mutations in oncogenes and/or tumor suppressor genes, and/or any other technique that changes the growth and/or immortalization properties of a cell.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, or the like.

As used herein, the term “metastasis” refers to a cancer which has spread to a site distant from the origin of the cancer, e.g. from the primary tumor. Sites of metastasis include without limitation, the bone, lymph nodes, lung, liver, and brain.

As used herein, the term “angiogenesis” refers to the development of blood vessels in a patient.

As used herein, the term “clinical endpoint” refers to a measurable event indicative of cancer. Clinical endpoints include without limitation, time to first metastasis, time to subsequent metastasis, size and/or number of metastases, size and/or number of tumors, location of tumors, aggressiveness of tumors, quality of life, pain and the like. Those skilled in the art are credited with the ability to determine and measure clinical endpoints. Methods of measuring clinical endpoints are known to those of skill in the art.

As used herein, the term “sample” refers to biological material from a patient The sample assayed by the present invention is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

As used herein, the term “biological molecule” includes, but is not limited to, polypeptides, nucleic acids, and saccharides.

As used herein, the term “modulating” refers to a change in the quality or quantity of a gene, protein, or any molecule that is inside, outside, or on the surface of a cell The change can be an increase or decrease in expression or level of the molecule. The term “modulates” also includes changing the quality or quantity of a biological ftmction/activity including, without limitation, cell proliferation, growth, adhesion, cell survival, apoptosis, intracellular signaling, cell-to-cell signaling, and the like.

As used herein, the term “modulator” refers to a composition that modulates one or more physiological or biochemical events associated with cancer. In some embodiments the modulator inhibits one or more biological activities associated with cancer. In some embodiments the modulator is a small molecule, an antibody, a mimetic, a decoy or an oligonucleotide. In some embodiments the modulator acts by blocking ligand binding or by competing for a ligand-binding site. In some embodiments the modulator acts independently of ligand binding. In some embodiments the modulator does not compete for a ligand binding site. In some embodiments the modulator blocks expression of a gene product involved in cancer. In some embodiments the modulator blocks a physical interaction of two or more biomolecules invohnd in cancer. In some embodiments modulators of the invention inhibit one or more FXYD5 biological activities selected from the group consisting of cancer cell growth, tumor formation, cancer cell proliferation, cancer cell survival, cancer cell metastasis, cell migration, angiogenesis, FXYD5 signaling FXYD5-mediated inhibition of cell-cell adhesion, cell-cell interaction, FXYD5-mediated cell-cell membrane interaction, FXYD5-mediated cell-extracellular matrix interaction, integrin mediated activities, FXYD5 surface expression, FXYD5-mediated cell-extracellular matrix degradation. In some embodiments the FXYD5 modulator inhibits FXYD5 expression.

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide, etc. Also encompassed by this term are biopolymeric products that are made using an RNA gene product as a template (i.e. cDNA of the RNA). A gene product may be made enzymatically, recombinantly, chemically, or within a cell to which the gene is native. In some embodiments, if the gene product is proteinaceous, it exhibits a biological activity. In some embodiments, if the gene product is a nucleic acid, it can be translated into a proteinaceous gene product that exhibits a biological activity.

“Modulation of FXYD5 activity”, as used herein, refers to an increase or decrease in FXYD5 activity that can be a result of, for example, interaction of an agent with a FXYD5 polynucleotide or polypeptide, inhibition of FXYD5 transcription and/or translation (e.g., through antisense or siRNA interaction with the FXYD5 gene or FXYD5 transcript, through modulation of transcription factors that facilitate FXYD5 expression), and the like. For example, modulation of a biological activity refers to an increase in a biological activity or a decrease in a biological activity). Modulation of FXYD5 activity that results in a decrease of FXYD5 activity is of particular interest in the present invention. In particular, FXYDS activity can be assessed by measuring FXYD5-mediated inhibition of cell adhesion. FXYD5 activity can also be assessed by means including without limitation, assaying Na,K-ATPase activity assessing FXYD5 polypeptide levels, or by assessing FXYD5 transcription levels. Comparisons of FXYD5 activity can also be accomplished by measuring levels of a FXYD5 downstream marker, measuring inhibition of FXYD5 signaling measuring activation of FXYDS mediated cancer cell apoptosis, measuring inhibition of cancer cell growth, and measuring inhibition of tumor formation.

As used herein, the term “inhibit” refers to a reduction, decrease, inactivation or down-regulation of an activity or quantity. For example, in the context of the present invention, FXYD5 modulators may inhibit one or more of cancer cell growth, tumor formation, cancer cell proliferation, cancer cell survival, cancer cell metastasis, cell migration, angiogenesis, FXYD5 signaling, FXYD5-mediated inhibition of cell-cell adhesion, cell-cell interaction, FXYD5-mediated cell-cell membrane interaction, FXYD5-mediated cell-extracellular matrix interaction, integrin mediated activities, cadherin-mediated activities, FXYD5 surface expression, and FXYD5 expression. Inhibition may be by at least 25%, at least 50%, at least 75%, at least 80%, at least 90%, at least 95%, at least 97%, at least 98%, at least 99%, or 100%, as compared to a control.

As used herein, the term “differentially expressed in a cancer cell” and “a polynucleotide that is differentially expressed in a cancer cell” are used interchangeably herein, and refer to a polynucleotide that represents or corresponds to a gene that is differentially expressed in a cancerous cell when compared with a cell of the same cell type that is not cancerous, e.g., mRNA is found at levels at least about 25%, at least about 50% to about 75%, at least about 90%, at least about 1.5-fold, at least about 2-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source, or between one cell in situ and a second cell removed from its tissue source. In some embodiments, FXYD5 is upregulated in the cancer cell as compared to the normal cell.

A FXYD5 associated-cancer is “inhibited” if at least one symptom of the cancer is alleviated, terminated, slowed, or prevented. As used herein, a FXYD5 associated-cancer is also “inhibited” if recurrence or metastasis of the cancer is reduced, slowed, delayed, or prevented.

As used herein, the phrase “modulation of FXYD5-mediated inhibition of cell adhesion” refers to modulation (e.g., increase) of cell-to-cell adhesion in the presence of a FXYD5 inhibitor wherein at least one cell differentially expresses FXYD5. In this context, FXYD5-mediated inhibition of cell adhesion can be decreased by a FXYD5 inhibitor by at least 25%, at least 50%, at least 75%, at least 85%, at least 90%, at least 95%, up to 100% relative to FXYD5-mediated inhibition of cell adhesion in the absence of a FXYD5 inhibitor. Comparisons of cell adhesion can be accomplished by measuring, for example, by labeling the cells of interest, incubating them with a population of unlabeled cells adhering to a substrate, and washing to separate the adherent from the non-adherent populations. In this =timer, cell adhesion is determined by measuring the amount of label retained on the substrate. Examples of assay systems include, but are not limited to labeling with fluorescent probes such as calcein AM, CFMDA (5-chloromethylfluorescein diacetate), 5(6)-CFDA-SE [5-(and-6)-carboxyfluorescein diacetate, suecinimidyl ester] and measuring fluorescence in fluorescence plate reader or via flow cytometry.

As used herein, the phrase “inhibits proliferation” refers to reducing or eliminating FXYDS-mediated proliferation and can be measured via a number of methods known to those of skill in the art Cell proliferation assays include, without limitation, MTT assays (for example, the Vybrant® MTT Cell Proliferation Assay Kit (Invitrogen)); BrdU incorporation assays (for example, the Absolute-S SBLP assay (Invitrogen)); measuring intracellular ATP levels (commercial versions of the assay include ATPLitena-M, 1,000 Assay Kit (PerkinElmer) and ATP Cell Viability Assay Kit (BioVision)); Di0c18 assay, a membrane permeable dye (Invitrogen); Glucose-6-phosphate dehydrogenase activity assay (for example, the Vibrant cytotoxicity assay (Invitrogen)); measuring cellular LDH activity; and ³H-thyimidine incorporation and the Cell Titer Glo Assay (Promega).

As used herein, the phrase “inhibits angiogenesis” refers to reducing or eliminating FXYD5-mediated angiogenesis. Angiogenesis can be detected via a number of methods known to those of skill in the art, including, without limitation, cell proliferation assays, cell migration assays, cell differentiation assays, organ culture (ex vivo) assays, chick chorioallantoic membrane (CAM) assays, corneal angiogenesis assays, Matrigel plug assays, and tumor volume assays in SCID mice, nude mice, or C57BL mice.

Cell migration assays include, without limitation, blind-well chemotaxis chamber, e.g., modified Boyden chamber and the Phagokinetic hack assay. Cell differentiation assays include, without limitation, tube formation in collagen, fibrin clots, or Matrigel, followed by electron microscopy. Organ culture (ex vivo) assays include, without limitation, rat aortic ring assay and chick aortic arch assay.

As used herein, the phrase “inhibits progression through the cell cycle” refers to slowing or stalling the cell division. Cell-cycle progression can be assayed by bromodeoxyuridine (BRDU) incorporation. Such assays identify a cell population undergoing DNA synthesis by incorporation of BRDU into newly synthesized DNA. Newly-synthesized DNA may then be detected using an anti-BRDU antibody (Hoshino et al., 1986, int. J. Cancer 38, 369; Campana et al., 1988, J. Immunol. Meth. 107, 79), or by other means. Cell proliferation can also be assayed by phospho-histone H3 staining, which identifies a cell population undergoing mitosis by phosphorylation of histone H3. Phosphorylation of histone H3 at serine 10 is detected using an antibody specific to the phosphorylated form of the serine 10 residue of histone H3. (Chadlee, D. N. 1995, J. Biol. Chem 270:20098-105). Cell proliferation can also be examined using [³H]-thymidine incorporation (Chen, J., 1996, Oncogene 13:1395-403; Jeoung, J., 1995, J. Biol. Chem. 270:18367-73). This assay allows for quantitative characterization of S-phase DNA synthesis. In this assay, cells synthesizing DNA will incorporate [³H]-thymidine into newly synthesized DNA. Incorporation can then be measured by standard techniques such as by counting of radioisotope in a scintillation counter (e.g., Beckman L S 3800 Liquid Scintillation Counter). Another proliferation assay uses the dye Alamar Blue (available from Biosource International), which fluoresces when reduced in living cells and provides an indirect measurement of cell number (Voytils-Harbin S L et al., 1998, In Vitro Cell Dev Biol Anim 34:239-46). Yet another proliferation assay, the MTS assay, is based on in vitro cytotoxicity assessment of industrial chemicals, and uses the soluble tetrazolium salt, MTS. MTS assays are commercially available and include the Promega CellTiter 96® AQueous Non-Radioactive Cell Proliferation Assay (Cat.# G5421). Cell proliferation can also be assayed by colony formation in soft agar (Sambrook et al., Molecular Cloning, Cold Spring Harbor (1989)). Cell proliferation may also be assayed by measuring ATP levels as indicator of metabolically active cells. Such assays are commercially available and include Cell Titer-Glo™ (Promega). Cell cycle proliferation can also be assayed by flow cytometry (Gray J Wet al. (1986) Int J Radiat Biol Relat Stud Phys Chem Med 49:237-55). Cells may be stained with propidium iodide and evaluated in a flow cytometer to measure accumulation of cells at different stages of the cell cycle.

As used herein, the phrase “increasing cancer cell apoptosis” refers to increasing apoptosis of cancer cells that differentially express FXYD5 in the presence of a FXYD5 inhibitor. In this context, cancer cell apoptosis can be increased by FXYD5 inhibitor by at least 25%, at least 50%, at least 75%, at least 85%, at least 90%, at least 95%, up to 100% relative to cancer cell apoptosis in the absence of a FXYD5 inhibitor. Comparisons of cancer cell apoptosis can be accomplished by measuring, for example, DNA fragmentation, caspase activity, loss of mitochondrial membrane potential, increased production of reactive oxygen species (ROS), intracellular acidification, chromatin condensation, phosphatidyl serine (PS) levels at the cell surface, and increased cell membrane permeability.

DNA fragmentation can be measured, for example, with the TUNEL assay (terminal deoxynucleotide transferase dUTP nick end labeling). Commercial versions of the assay are widely available, for example, APO-BrdU™ TUNEL Assay Kit (Invitrogen), APO-DIRECT™ Kit (BD Biosciences Pharmingen) and ApoAlert™ DNA Fragmentation Assay Kit (Clontech, a Takers Bio Company).

Caspase activity can be monitored via fluorogenic, chromogenic and luminescent substrates specific for particular caspases. Commercial assay kits are available for at least caspases 1, 2, 3, 6, 7, 8 and 9. (See, for example, Invitrogen, Chemicon, CalBiochem, BioSource International, Biovision).

Loss of mitochondrial membrane potential can be measured with fluorescent dyes that differentially accumulate in healthy active mitochondria. One non-limiting example is the MitoTracker Red system from Invitrogen.

Production of reactive oxygen species (ROS) can be measured with fluorescent dyes including, for example, H2DCFDA (Invitrogen).

Intracellular acidification can be measured with fluorescent or chromogenic dyes.

Chromatin condensation can be measured with fluorescent dyes including, for example, Hoechst 33342.

Phosphatidyl serine (PS) levels can be measured at the cell surface. For example, Annexin V has a high affinity for PS. Numerous commercially available assays are suitable to monitor the binding of labeled AnnexinV to the cell surface.

Cell membrane permeability can be measured using dyes, such as the fluorescent dye, YO-PRO-1 (Invitrogen) which can enter apoptotic, but not necrotic cells.

As used herein, the phrase “inhibits cancer cell growth” refers to inhibition or abolition of cancer cell growth in the presence of a FXYDS inhibitor wherein the cell differentially expresses FXYD5. In this context, cancer cell growth can be decreased by FXYD5 inhibitor by at least 25%, at least 50%, at least 75%, at least 85%, at least 90%, at least 95%, up to 100% relative to cancer cell growth in the absence of a FXYD5 inhibitor. Comparisons of cancer cell growth can be accomplished using, for example, WI* assay (for example, the Vybrant® MTT Cell Proliferation Assay Kit (Invitrogen)); BrdU incorporation (for example, the Absolute-S SBIP assay (Invitrogen)); measuring intracellular ATP levels (for example using ATPLite™-M, 1,000 Assay Kit (PerkinElmer) or ATP Cell Viability Assay Kit (BioVision)); DiOc18 assay, a membrane permeable dye (Invitrogen); Glucose-6-phosphate dehydrogenase activity assay (for example, the Vibrant cytotoxicity assay (Invitrogen)); or measuring cellular LDH activity.

As used herein, the phrase “inhibits tumor formation” refers to inhibition or abolition of tumor formation in the presence of a FXYD5 inhibitor wherein the tumor comprises cells that differentially express FXYD5. In this context, tumor formation can be decreased by a FXYDS inhibitor by at least 25%, at least 50%, at least 75%, at least 85%, at least 90%, at least 95%, and up to 100% relative to tumor formation in the absence of a FXYD5 inhibitor. Comparisons of tumor formation can be accomplished using, for example, cell based assays (for example colony formation in soft agar); in vivo models of tumor formation typically relying upon injecting the cells of interest into animals (for example, athymic mice or rats, irradiated mice or rats; inoculation into immunologically privileged sites such as brain, cheek pouch or eye; inoculation of syngeneic animals), and monitoring the size of the mass after a defined time period.

As used herein, the phrase “inhibits cancer cell survival” refers to the inhibition of survival of cancer cells that express FXYDS. In some embodiments the term refers to effecting apopotosis of cancer cells that express FXYD5. In this context, survival of FXYD5-expressing cancer cells can be decreased by an inhibitory agent by at least 25%, at least 50%, at least 75%, at least 85%, at least 90%, at least 95%, up to 100% relative to cancer cell survival in the absence of a FXYD5 inhibitor and/or in a normal cell.

As used herein, the phrase “modulates FXYD5 inhibition of cell-cell interaction” refers to increasing an interaction between two or more cells that express FXYD5. In some embodiments, the interaction between the cells leads to a cell signal. Cell-cell interaction can be detected via a number of methods known to those of skill in the art, including, without limitation, the observation of membrane exchange between co-cultured, pre-labeled cells, labeled, for example, with different fluorescent membrane stains including PKH26 and PKH67 (Sigma).

A “FXYD5 downstream marker”, as used herein, is a gene or activity which exhibits altered level of expression in a cancer tissue or cancer cell compared to the level of expression by the gene or activity in normal or healthy tissue, or is a property altered in the presence of a FXYD5 modulator (e.g., cell adhesion). In some embodiments, the downstream markers exhibit altered levels of expression when FXYD5 is perturbed with a FXYDS modulator of the present invention. In various embodiments, E-cadherin activity and/or beta-catenin localization employed as a downstream marker of FXYD5 activity. For example, decreased E-cadherin activity or expression can be indicative of increased FXYD5 expression or activity. Increased nuclear localization of beta-catenin can also be indicative of increased FXYD5 expression or activity.

As used herein, the term “up-regulates” refers to an increase, activation or stimulation of an activity or quantity. Up-regulation may be by at least 25%, at least 50%, at least 75%, at least 100%, at least 150%, at least 200%, at least 250%, at least 400%, or at least 500% as compared to a control.

As used herein, the term “N-terminus” refers to the first 10 amino acids of a protein.

As used herein, the term “C-terminus” refers to the last 10 amino acids of a protein.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

As used herein, the term “extracellular domain” refers to the portion of a molecule that is outside or external to a cell. In the context of the present invention, an N-terminal extracellular domain refers to the extracellular domain that is present at the N-terminal of the molecule immediately before the first transmembrane domain.

As used herein, the term “ligand binding domain” refers to any portion or region of a receptor retaining at least one qualitative binding activity of a corresponding native sequence of FXYD5.

The term “region” refers to a physically contiguous portion of the primary structure of a biomolecule. In the case of proteins, a region is defined by a contiguous portion of the amino acid sequence of that protein. In some embodiments a “region” is associated with a function of the biomolecule.

The term “fragment” as used herein refers to a physically contiguous portion of the primary structure of a biomolecule. In the case of proteins, a portion is defined by a contiguous portion of the amino acid sequence of that protein and refers to at least 3-5 amino acids, at least 8-10 amino acids, at least 11-15 amino acids, at least 17-24 amino acids, at least 25-30 amino acids, and at least 30-45 amino acids. In the case of oligonucleotides, a portion is defined by a contiguous portion of the nucleic acid sequence of that oligonucleotide and refers to at least 9-15 nucleotides, at least 18-30 nucleotides, at least 33-45 nucleotides, at least 48-72 nucleotides, at least 75-90 nucleotides, and at least 90-130 nucleotides. In some embodiments, portions of biomolecules have a biological activity. In the context of the present invention, FXYD5 polypeptide fragments do not comprise an entire FXYD5 polypeptide sequence. In some embodiments, FXYD5 fragments retain one or more activities of native, full-length FXYD5.

As used herein, the phrase “FXYD5-related cells/tumors/samples” and the like refers to cells, samples, tumors or other pathologies that are characterized by differential expression of FXYD5 relative to non-cancerous and/or non-metastatic cells, samples, tumors, or other pathologies. In some embodiments, FXYD5-related cells, samples, tumors or other pathologies are characterized by increased evidence of FXYD5 expression relative to non-metastatic cells, samples, tumors, or other pathologies.

As used herein, the term “antibody” refers to monoclonal and polyclonal antibodies, single chain antibodies, chimeric antibodies, bifunctionallhispecific antibodies, humanized antibodies, human antibodies, and complementary determining region (CDR)-grafted antibodies, that are specific for the target protein or fragments thereof. The term “antibody” further includes in vivo therapeutic antibody gene transfer. Antibody fragments, including Fab, Fab′, F(ab′)2, scFv, and Fv are also provided by the invention.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks at al., J. MoL Biol., 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of an intact antibody, in some embodiments comprising the antigen-binding or variable region thereof. Examples of antibody fragments include Fab, Fab′, F(ab′)2, and Fv fragments; diabodies; linear antibodies (Zapata et al., Protein Beg. 8(10):1057-1062 [1995]); single-chain antibody molecules; and multispecific antibodies formed from antibody fragment(s).

An “intact” antibody is one that comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof Preferably, the intact antibody has one or more effector functions.

Antibody “effector functions” refer to those biological activities attributable to the Fc region (a native sequence Fc region or amino acid sequence variant Fc region) of an antibody. Examples of antibody effector functions include, without limitation, Clq binding complement dependent cytotoxicity, Fe receptor binding; antibody-dependent cell-mediated cytotoxicity (ADCC); phagocytosis; down regulation of cell surface receptors (e.g. B cell receptor, BCR).

“Antibody-dependent cell-mediated cytotoxicity” or “ADCC” refers to a fonn of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen- bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcyRI, FcγRI and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Human effector cells” are leukocytes that express one or more FcRs and perform effector functions. Preferably, the cells express at least FcγRIII and perform ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof; e.g. from blood or PBMCs as described herein.

The terms “Fc receptor” or “FcR” are used to describe a receptor that binds to the Fe region of an antibody. The preferred FcR is a native sequence human FcR. Moreover, a preferred FcR is one that binds an IgG antibody (a gamma receptor) and includes receptors of the FcγRI, FcγRII, and FcyγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immtmoreceptor tyrosine-based activation motif (ITAM) in its cytoplasmic domain. Inhibiting receptor FcyRIIB contains an iramunoreceptor tyrosine-based inhibition motif (ITIM) in its cytoplasmic domain. (see review M. in Daron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas at at, J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetes (Guyer at al., J. Immunol. 117:587 (1976) and Kim at al., J. Immunol. 24:249 (1994)).

“Complement dependent cytotoxicity” or “CDC” refers to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro at at, J. Immunol. Methods 202:163 (1996), may be performed.

As used herein, the term “epitope” refers to an antigenic determinant of a polypeptide. In some embodiments an epitope may comprise 3 or more amino acids in a spatial conformation which is unique to the epitope. In some embodiments epitopes are linear or conformational epitopes. Generally an epitope consists of at least 4, at least 6, at least 8, at least 10, and at least 12 such amino acids, and more usually, consists of at least 8-10 such amino acids. Methods of determining the spatial conformation of amino acids are known in the art, and include, for example, x-ray crystallography and 2-dimensional nuclear magnetic resonance.

The term “antagonist” is used in the broadest sense, and includes any molecule that partially or fully blocks, inhibits, or neutralizes a biological activity of a tumor cell antigen disclosed herein. In a similar manner, the term “agonist” is used in the broadest sense and includes any molecule that mimics a biological activity of a tumor cell antigen disclosed herein. Suitable agonist or antagonist molecules specifically include agonist or antagonist antibodies or antibody fragments, fragments or amino acid sequence variants of tumor cell antigens, peptides, antisense oligonucleotides, small organic molecules, etc. Methods for identifying agonists or antagonists of a tumor cell antigen may comprise contacting a tumor cell expressing the antigen of interest with a candidate agonist or antagonist molecule and measuring a detectable change in one or more biological activities normally associated with the tumor cell antigen. The antagonist may also be a peptide generated by rational design or by phage display (see, e.g., WO98/35036 published 13 Aug. 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

As used herein, the term “oligonucleotide” refers to a series of linked nucleotide residues. Oligonucleotides include without limitation, antisense and sIRNA oligonucleotides. Oligonucleotides comprise portions of a DNA sequence and have at least about 10 nucleotides and as many as about 500 nucleotides. In some embodiments oligonucleotides comprise from about 10 nucleotides to about 50 nucleotides, from about 15 nucleotides to about 30 nucleotides, and from about 20 nucleotides to about 25 nucleotides. Oligonucleotides may be chemically synthesized and can also be used as probes. In some embodiments oligonucleotides are single stranded. In some embodiments oligonucleotides comprise at least one portion which is double stranded. In some embodiments the oligonucleotides are antisense oligonucleotides (ASO). In some embodiments the oligonucleotides are RNA interference oligonucleotides (RNAi oligonucleotides).

As used herein, the term “antisense oligonucleotide” refers to an unmodified or modified nucleic acid having a nucleotide sequence complementary to a FXYD5 polynucleotide sequence including polynucleotide sequences associated with the transcription or translation of FXYD5 (e.g., a promoter of a FXYD5 polynucleotide), where the antisense polynucleofide is capable of hybridizing to a FXYD5 polynucleotide sequence. Of particular interest are antisense polynucleotides capable of inhibiting transcription and/or translation of FXYD5 polypeptide-encoding polynucleotide either in vitro or in vivo.

As used herein, the terms “siRNA oligonucleotides”, “RNAi oligonucleotides”, “short interfering RNA”, or “siRNA” are used interchangeably and refer to oligonucleotides that work through post-transcriptional gene silencing, also known as RNA interference (RNAi). The terms refer to molecules capable of RNA interference “RNAi”, (see Kreutzer et al., WO 00/44895; Zernicka-Goetz at al. WO 01/36646; Fire, WO 99/32619; Mello and Fire, WO 01/29058). SIRNA molecules are generally RNA molecules but further encompass chemically modified nucleotides and non-nucleotides. SiRNA gene-targeting experiments have been carried out by transient snRNA transfer into cells (achieved by such classic methods as liposome-mediated transfection, electroporation, or microinjection). Molecules of siRNA are 21- to 23-nucleotide RNAs, with characteristic 2- to 3-nucleotide 3′-overhanging ends resembling the RNase III processing products of long double-stranded RNAs (dsRNAs) that normally initiate RNAi.

As used herein, the term “therapeutically effective amount” is meant to refer to an amount of a medicament which produces a medicinal effect observed as reduction or reverse in one or more clinical endpoints, growth and/or survival of cancer cell, or metastasis of cancer cells in an individual when a therapeutically effective amount of the medicament is administered to the individual. Therapeutically effective amounts are typically determined by the effect they have compared to the effect observed when a composition which includes no active ingredient is administered to a similarly situated individual. The precise effective amount for a subject will depend upon the subject's size and health, the nature and extent of the condition, and the therapeutics or combination of therapeutics selected for administration. However, the effective amount for a given situation is determined by routine experimentation and is within the judgment of the clinician.

As used herein, the terms “in combination with” or “in conjunction with” refer to administration of the FXYD5 modulators of the invention with other therapeutic regimens.

As used herein, the term “susceptible” refers to patients for whom FXYD5 therapy is an acceptable method of treatment, i.e., patients who are likely to respond positively. Cancer patients susceptible to FXYD5 therapy express high levels of FXYD5 relative to those patients not susceptible to FXYD5 therapy. Cancer patients who are not good candidates for FXYD5 therapy include cancer patients with tumor samples that lack or have lower levels of FXYD5 in or on their cancer cells.

As used herein, the term “detecting” means to establish, discover, or ascertain evidence of an activity (for example, gene expression) or biomolecule (for example, a polypeptide).

A “native sequence” polypeptide is one that has the same amino acid sequence as a polypeptide derived from nature. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “amino acid sequence variant” refers to polypeptides having amino acid sequences that differ to some extent from a native sequence polypeptide. Ordinarily, amino acid sequence variants will possess at least about 70%, at least 80%, at least 90%, at least 95%, at least 98%, and at least 99% homology with at least one receptor binding domain of a native ligand or with at least one ligand binding domain of a native receptor. The amino acid sequence variants possess substitutions, deletions, and/or insertions at certain positions within the amino acid sequence of the native amino acid sequence.

As used herein, the phrase “homologous nucleotide sequence,” or “homologous amino acid sequence,” or variations thereof; refers to sequences characterized by a homology, at the nucleotide level or amino acid level, of at least a specified percentage and is used interchangeably with “sequence identity”. Homologous nucleotide sequences include those sequences coding for isoforms of proteins. Such isofonns can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals.

Homologous nucleotide sequences also include, but are not limited to, naturally occurring allelic variations and mutations of the nucleotide sequences set forth herein. Homologous amino acid sequences include those amino acid sequences which contain conservative amino acid substitutions and which polypeptides have the same binding and/or activity. In some embodiments, a nucleotide or amino acid sequence is homologous if it has at least 80%, 85%, 90%, 95%, 96%, 97%, 98%, or 99% sequence identity to the wild-type sequence. In some embodiments, a nucleotide or amino acid sequence is homologous if it has 1-10, 10-20, 20-30, 30-40, 40-50, or 50-60 nucleotide/amino acid substitutions, additions, or deletions. In some embodiments, the homologous amino acid sequences have no more than 5 (e.g. 5 or fewer) or no more than 3 (e.g. 3 or fewer) conservative amino acid substitutes. Homologous amino acid sequences also include those amino acid sequences which contain conservative amino acid substitutions and which polypeptides have the same binding and/or activity as native FXYD5. In some embodiments, altered expression levels of FXYD5 homologs are indicative of cancer.

Percent homology or identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, homology between the probe and target is between about 70% to about 80%. In some embodiments, nucleic acids have nucleotides that are about 85%, about 90%, about 92%, about 94%, about 95%, about 97%, about 98%, about 99% and about 100% homologous to SEQ ID NO:1, or a portion thereof

Homology may also be at the polypeptide level. In some embodiments, polypeptides are about 80%, about 85%, about 90%, about 92%, about 94%, about 95%, about 97%, about 98%, about 99% and about 100% homologous to SEQ ID NO:2, or a portion thereof.

As used herein, the term “probe” refers to nucleic acid sequences of variable length. In some embodiments probes comprise at least about 10 ,and as many as about 6,000 nucleotides. In some embodiments probes comprise at least 12, at least 14, at least 16, at least 18, at least 20, at least 25, at least 50 or at least 75 consecutive nucleotides. Probes are used in the detection of identical, similar, or complementary nucleic acid sequences. Longer length probes are usually obtained from natural or recombinant sources, are highly specific to the target sequence, and are much slower to hybridize to the target than are oligomers. Probes may be single- or double-stranded and are designed to have specificity in PCR, hybridization membrane-based, in situ hybridization (ISH), fluorescent in situ hybridization (FISH), or ELISA-like technologies.

As used herein, the term “mixing” refers to the process of combining one or more compounds, cells, molecules, and the like together in the same area. This may be performed, for example, in a test tube, petri dish, or any container that allows the one or more compounds, cells, or molecules, to be mixed.

As used herein the term “isolated” refers to a polynucleotide, a polypeptide, an antibody, or a host cell that is in an environment different from that in which the polynucleotide, the polypeptide, or the antibody naturally occurs. Methods of isolating cells are well known to those skilled in the art. A polynucleotide, a polypeptide, or an antibody which is isolated is generally substantially purified.

As used herein, the term “substantially purified” refers to a compound (e.g., either a polynucleotide or a polypeptide or an antibody) that is removed from its natural environment and is at least 60% free, at least 75% free, and at least 90% free from other components with which it is naturally associated.

As used herein, the term “binding” means the physical or chemical interaction between two or more biomolecules or compounds. Binding includes ionic, non-ionic, hydrogen bonds, Van der Waals, hydrophobic interactions, etc. Binding can be either direct or indirect; indirect being through or due to the effects of another biomolecule or compound. Direct binding refers to interactions that do not take place through or due to the effect of another molecule or compound but instead are without other substantial chemical intermediates.

As used herein, the term “contacting” means bringing together, either directly or indirectly, one molecule into physical proximity to a second molecule. The molecule can be in any number of buffers, salts, solutions, etc. “Contacting” includes, for example, placing a polynucleotide into a beaker, microtiter plate, cell culture flask, or a microarray, or the hle, which contains a nucleic acid molecule. Contacting also includes, for example, placing an antibody into a beaker, microtiter plate, cell culture flask, or microarray, or the like, which contains a polypeptide. Contacting may take place in vivo, ex vivo, or in vitro.

As used herein, the phrase “stringent hybridization conditions” or “stringent conditions” refers to conditions under which a probe, primer, or oligonucleotide will hybridize to its target sequence, but to a minimal number of other sequences. Stringent conditions are sequence-dependent and will be different in different circumstances. Longer sequences will hybridize with specificity to their proper complements at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tₘ, 50% of the probes are hybridized to their complements at equilibrium. Typically, stringent conditions will be those in which the salt concentration is less than about 1.0 M sodium ion, typically about 0.01 to 1.0 M sodium ion (or other salts) at pH 7.0 to 83 and the temperature is at least about 30° C. for short probes, primers or oligonucleotides (e.g., 10 to 50 nucleotides) and at least about 60° C. for longer probes, primers or oligonucleotides. Stringent conditions may also be achieved with the addition of destabilizing agents, such as formamide.

As used herein, the term “moderate stringency conditions” refers to conditions under which a probe, primer, or oligonucleotide will hybridize to its target sequence, but to a limited number of other sequences. Moderate conditions are sequence-dependent and will be different in different circumstances. Moderate conditions are well-known to the art skilled and are described in, inter alis, Manitatis et al. (Molecular Cloning: A Laboratory Manual, Cold Spring Harbor Laboratory; 2nd Edition (December 1989)).

The nucleic acid compositions described herein can be used, for example, to produce polypeptides, as probes for the detection of mRNA in biological samples (e.g., extracts of human cells) or cDNA produced from such samples, to generate additional copies of the polynucleotides, to generate ribozymes or oligonucleotides (single and double stranded), and as single stranded DNA probes or as triple-strand forming oligonucleotides. The probes described herein can be used to, for example, determine the presence or absence of the polynucleotides provided herein in a sample. The polypeptides can be used to generate antibodies specific for a polypeptide associated with cancer, which antibodies are in turn useful in diagnostic methods, prognostic methods, and the hie as discussed in more detail herein. Polypeptides are also useful as targets for therapeutic intervention, as discussed in more detail herein. Antibodies of the present invention may also be used, for example, to purify, detect, and target the polypeptides of the present invention, including both in vitro and in vivo diagnostic and therapeutic methods. For example, the antibodies are useful in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988). These and other uses are described in more detail below.

As used herein the term “imaging agent” refers to a composition linked to an antibody, small molecule, or probe of the invention that can be detected using techniques known to the art-skilled. As used herein, the term “evidence of gene expression” refers to any measurable indicia that a gene is expressed.

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the ₚroduction of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the lilre, can also be present in such vehicles.

Specific examples of cancers that can be treated by the methods and compositions of the present invention include, but are not limited to, FXYD5 associated cancers. As used herein, “FXYD5 associated cancer” refers to a cancer characterized by cells that differentially express FXYD5 relative to non-cancerous cells. The present invention is also applicable to any tumor cell-type where FXYD5 plays a role in cancer cell growth, tumor formation, cancer cell proliferation, cancer cell metastasis, cell migration, angiogenesis, FXYD5 signaling, cell-cell adhesion, cell-cell interaction, FXYD5-mediated cell-cell membrane interaction, FXYD5-mediated cell-extracellular matrix interaction, integrin mediated activities, FXYD5 surface expression, and FXYD5 expression. In some embodiments, the cancer is colon cancer, breast cancer, skin cancer, esophageal cancer, liver cancer, pancreatic cancer, prostatic cancer, uterine cancer, cervical cancer, lung cancer, bladder cancer, ovarian cancer, multiple myeloma and melanoma. In some embodiments, the cancer is ER-positive breast cancer. In some embodiments, the cancer is ER-negative breast cancer. In some embodiments, such cancers exhibit differential expression of FXYD5 of at least about 25%, at least about 50%, at least about 75%, at least about 100%, at least about 150%, at least about 200%, or at least about 300% as compared to a control.

The present invention provides methods and compositions that provide for the treatment, inhibition, and management of diseases and disorders associated with FXYD5 overexpression as well as the treatment, inhibition, and management of symptoms of such diseases and disorders. Some embodiments of the invention relate to methods and compositions comprising compositions that treat, inhibit or manage cancer including, without limitation, cancer metastases, cancer cell proliferation, cancer cell growth and cancer cell invasion.

The present invention further provides methods including other active ingredients in combination with the FXYD5 modulators of the present invention. In some embodiments, the methods further comprise administering one or more conventional cancer therapeutics to the patient In some embodiments the methods of the present invention further comprise treating the patient with one or more of chemotherapy, radiation therapy or surgery. For example, in some embodiments the patient is treated with methotrexate and/or doxorubicine in combination with the FXYD5 modulator.

The present invention also provides methods and compositions for the treatment, inhibition, and management of cancer or other hyperproliferative cell disorder or disease that has become partially or completely refractory to current or standard cancer treatment, such as surgery, chemotherapy, radiation therapy, hormonal therapy, and biological therapy.

The invention also provides diagnostic end/or imaging methods using the FXYD5 modulators of the invention, particularly FXYD5 antibodies, to diagnose cancer and/or predict cancer progression. In some embodiments, the invention provides methods of imaging and localizing tumors and/or metastases and methods of diagnosis and prognosis. In some embodiments, the invention provides methods for evaluating the appropriateness of FXYD5-related therapy.

FXYD5 Modulators

The present invention provides FXYD5 modulators for, inter alia, the treatment, diagnosis, detection or imaging of cancer. FXYD5 modulators are also useful in the preparation of medicaments for the treatment of cancer.

In some embodiments, the FXYD5 modulator is an oligonucleotide, a small molecule, a mimetic, a decoy, or an antibody. In some embodiments, the FXYD5 modulator inhibits a FXYD5 biological activity by at least 25%, 50%, 75%, 80%, 90%, 95%, 97%, 98%, 99% or 100%, as compared to a control. In some embodiments, the FXYD5 modulator inhibits FXYD5 expression by at least 25%, 50%, 75%, 80%, 90%, 95%, 97%, 98%, 99% or 100%, as compared to a control.

Antibodies

In some embodiments the FXYD5 modulator is a monoclonal antibody, a polyclonal antibody, a chimeric antibody, a human antibody, a humanized antibody, a single-chain antibody, or a Fab fragment. The antibody may be labeled with, for example, an enzyme, radioisotope, or fluorophore. In some embodiments the antibody has a binding affinity less than about 1×10⁵ Ka for a polypeptide other than FXYD5. In some embodiments, the FXYD5 modulator is a monoclonal antibody which binds to FXYD5 with an affinity of at least 1×10⁸ Ka.

The invention also provides antibodies that competitively inhibit binding of an antibody to an epitope of the invention as determined by any method known in the art for determining competitive binding using, for example, immunoassays. In some embodiments, the antibody competitively inhibits binding to the epitope by at least 95%, at least 90%, at least 85%, at least 80%, at least 75% or at least 50%.

In some embodiments the antibody is a humanized antibody. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin, loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is descnbed, for example, in U.S. Pat. Nos. 5,545,807; 5,545,806; 5,569,825; 5,625,126; 5,633,425; 5,661,016, and in the following scientific publications: Marks et al., Bioneclmology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); Jones at al., Nature 321:522-525 (1986); Morrison at al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immure 28:489-498 (1991); Padlan, Molec. Immunol. 31(3):169-217 (1994); and Kettleborough, C. A. et al., Protein Eng. 4(7):773-83 (1991) each of which is incorporated herein by reference.

Antibodies of the present invention may function through different mechanisms. In some embodiments, antibodies trigger antibody-dependent cellular cytotoxicity (ADCC), a lytic attack on antibody-targeted cells. In some embodiments, antibodies have multiple therapeutic functions, including, for example, antigen-binding, induction of apoptosis, and complement-dependent cellular cytotoxicity (CDC).

In some embodiments, antibodies of the present invention may act as agonists or antagonists of the polypeptides of the present invention. For example, in some embodiments the present invention provides antibodies which disrupt the interaction between FXYD5 and a ligand, either partially or fully. In some embodiments antibodies of the present invention bind an epitope disclosed herein, or a portion thereof. In some embodiments, antibodies are provided that modulate ligand activity or receptor activity by at least 95%, at least 90%, at least 85%, at least 80%, at least 75% or at least 50% compared to the activity in the absence of the antibody.

In some embodiments, the FXYDS antibodies mediate one or more of the following activities: up-regulation of E-cadherin expression or activity, modulation of Na/ATPase activity, inhibition of cancer cell growth, inhibition of tumor formation, inhibition of cancer cell survival, inhibition of cancer cell proliferation, inhibition of cancer cell metastasis, inhibition of cell migration, inhibition of FXYD5-mediated signaling, increased cell-cell adhesion, modulation of actin, inhibition of FXYD5 binding to an FXYD5 ligand, inhibition of angiogenesis, inhibition of cellular interactions with extracellular matrix, and upregulation of cancer cell apoptosis.

In some embodiments the present invention provides neutralizing antibodies. In some embodiments the neutralizing antibodies act at receptor antagonists, Le., inhibiting either all or a subset of the biological activities of the ligand-mediated receptor activation. In some embodiments the antibodies may be specified as agonists, antagonists or inverse agonists for biological activities comprising the specific biological activities of the peptides, of the invention disclosed herein.

The antibodies of the present invention may be used either alone or in combination with other compositions, such as chemotherapeutic agents. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

In addition to chimeric and humanized antibodies, fully human antibodies can be derived from transgenic mice having human immunoglobulin genes (see, e.g., U.S. Pat. Nos. 6,075,181, 6,091,001, and 6,114,598, all of which are incorporated herein by reference), or from phage display libraries of human immunoglobulin genes (see, e.g. McCafferty et al., Nature, 348:552-554 (1990). Clackson et al., Nature, 352:624-628 (1991), and Marks et al., J. Mol. Biol., 222:581-597 (1991)). In some embodiments, antibodies may be produced and identified by scFv-phage display libraries. Antibody phage display technology is available from commercial sources such as from Xoma (Berkeley, Calif.).

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial or limiting dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

As an alternative to the use of hybridomas for expression, antibodies can be produced in a cell line such as a CHO or myeloma cell lines, as disclosed in U.S. Pat. Nos. 5,545,403; 5,545,405; and 5,998,144; each incorporated herein by reference. Briefly the cell line is transfected with vectors capable of expressing a light chain and a heavy chain, respectively. By transfecting the two proteins on separate vectors, chimeric antibodies can be produced. Immunol. 147:8; Banchereau et al. (1991) Clin. Immunol. Spectrum 3:8; and Banchereau et al. (1991) Science 251:70; all of which are herein incorporated by reference.

Human antibodies can also be produced using techniques known in the art, including phage display libraries [Hoogenboom and Winter, J. Mol. Biol., 227:381 (1991); Marks et at, J. Mol. BioL, 222:581 (1991)]. The techniques of Cole et al. and Boemer et aL are also available for the preparation of human monoclonal antibodies [Cole et al., Monoclonal Antibodies and Cancer Therapy, Alan R. Liss, p. 77 (1985) and Boemer et al., J. Immunol., 147(1):86 95 (1991)]. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779 783 (1992); Lonberg et al., Nature 368 856 859 (1994); Morrison, Nature 368, 812 13 (1994); Fishwild et al., Nature Biotechnology 14, 845 51 (1996); Neuberger, Nature Biotechnology 14, 826 (1996); Lonberg and Huszar, Intern. Rev. Immunol. 13 65 93 (1995); Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immure 28:489-498 (1991); Padlan, Molec. Immunol. 31(3):169-217 (1994); and Kettleborough, C A. et al., Protein Eng. 4(7):773-83 (1991) each of which is incorporated herein by reference. Fully humanized antibodies can be identified in screening assays using commercial resources such as Morphosys (Martinsried/Planegg, Germany).

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089, which are incorporated herein by reference.

Humanized antibodies can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the CrelLox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Antibodies of the present invention can also be produced using human engineering techniques as discussed in U.S. Pat. 5,766,886, which is incorporated herein by reference.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the hire are known in the art, and are used in immunization of for example, a transgenic mouse as described in WO 96/33735. The monoclonal antibodies can be tested for the ability to inhibit or neutralize the biological activity or physiological effect of the corresponding protein.

Antibodies of the present invention may be administered to a subject via in vivo therapeutic antibody gene transfer as discussed by Fang et al. (2005), Nat. Bioteclmol. 23, 584-590. For example recombinant vectors can be generated to deliver a multicistronic expression cassette comprising a peptide that mediates enzyme independent, cotranslational self cleavage of polypeptides placed between MAb heavy and light chain encoding sequences. Expression leads to stochiometric amounts of both MAb chains. A preferred example of the peptide that mediates enzyme independent, cotranslational self cleavage is the foot-and-mouth-disease derived 2A peptide.

Fragments of the antibodies are suitable for use in the methods of the invention so long as they retain the desired affinity of the full-length antibody. Thus, a fragment of an anti-FXYD5 antibody will retain the ability to bind to FXYD5. Such fragments are characterized by properties similar to the corresponding full-length anti-FXYD5 antibody, that is, the fragments will specifically bind a human FXYD5 antigen expressed on the surface of a human cell.

In some embodiments, the antibodies bind to one or more epitopes in an extracellular domain of FXYD5. In some embodiments, the antibodies modulate one or more FXYD5 related biological activities. In some embodiments the antibodies inhibit one or more of cancer cell growth, tumor formation, and cancer cell proliferation.

In some embodiments the antibody is a monoclonal antibody which binds to one or more FXYD5 epitopes in the extracellular domain.

Suitable antibodies according to the present invention can recognize linear or conformational epitopes, or combinations thereof.

Methods of predicting other potential epitopes to which an antibody of the invention can bind are well-known to those of sldll in the art and include without limitation, Kyte-Doolittle Analysis (Kyte, J. and Dolittle, R. F., J. Mol. Biol. (1982) 157:105-132), Hopp and Woods Analysis (Hopp, T. P. and Woods, ER., Proc. Natl. Acad. Sci. USA (1981) 78:3824-3828; Hopp, T. J. and Woods, KR., Mol. Immunol. (1983) 20:483-489; Hopp, T. J., J. Immunol. Methods (1986) 88:1-18.), Jameson-Wolf Analysis (Jameson, B A. and Wolf; H., Comput. Appl. Biosci. (1988) 4:181-186.), and Emini Analysis (Emini, E A., Schlief, W A., Colonno, R. I. and Wimmer, E., Virology (1985) 140:13-20).

In some embodiments, potential epitopes are identified by determining theoretical extracellular domains. Analysis algorithms such as TMpred (see K. Hofmann & W. Stoffel (1993) TMbase—A database of membrane spanning proteins segments Biol. Chem. Hoppe-Seyler 374,166) or TMHMM (A. Krogh, B. Larsson, G. von Heijne, and E L. L Sonnhammer. Predicting transmembrane protein topology with a hidden Markov model: Application to complete genomes. Journal of Molecular Biology, 305(3):567-580, January 2001) can be used to make such predictions. Other algorithms, such as SignalP 3.0 (Bednsten et al, (2004) J Mol Biol. 2004 Jul 16;340(4):783-95) can be used to predict the presence of signal peptides and to predict where those peptides would be cleaved from the full-length protein. The portions of the proteins on the outside of the cell can serve as targets for antibody interaction.

Antibodies are defined to be “specifically binding” if 1) they exhibit a threshold level of binding activity, and/or 2) they do not significantly cross-react with known related polypeptide molecules. The binding affinity of an antibody can be readily determined by one of ordinary skill in the art, for example, by Scatchard analysis (Scatchard, Ann. NY Acad. Sci. 51: 660-672, 1949). In some embodiments the antibodies of the present invention bind to their target epitopes or mimetic decoys at least 1.5-fold, 2-fold, 5-fold 10-fold, 100-fold, 10³-fold, 10⁴-fold, 10⁶-fold, 10⁶-fold or greater for the target cancer-associated polypeptide.

In some embodiments the antibodies bind with high affinity of 10⁻⁴ M or less, 10⁻⁷ M or less, 10⁴ M or less or with subnanomolar affinity (0.9, 0.8, 0.7, 0.6, 0.5, 0.4, 0.3, 0.2, 0.1 nM or even less). In some embodiments the binding affinity of the antibodies for FXYD5 is at least 1×10⁶ Ka. In some embodiments the binding affinity of the antibodies for FXYD5 is at least 5×10⁶ Ka, at least 1×10⁷ Ka, at least 2×10⁷ Ka, at least 1×10⁸ Ka, or greater. Antibodies of the present invention may also be described or specified in terms of their binding affinity to a polypeptide of the invention. In some embodiments binding affinities include those with a Kd less than 5×10⁻² M, 10⁻² M, 5×10⁻³ M, 10⁻³ M, 5×10⁻⁴ M, 10⁻⁴ M, 5×10⁻⁴ M, 10⁻⁴ M, 5×10⁻⁴ M, 10⁻⁴ M, 5×10⁻⁷ M, 10⁻⁷ M, 5×10⁻⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the antibodies of the present invention do not bind to known related polypeptide molecules, for example, if they bind FXYD5 polypeptide but not known related polypeptides (e.g., other FXYD family member polypeptides) using a standard Western blot analysis (Ausubel et al.).

In some embodiments, the antibodies of the present invention bind to orthologs, homologs, paralogs or variants, or combinations and subcombinations thereof, of FXYD5. In some embodiments, the antibodies of the present invention do not bind to orthologs, homologs, paralogs or variants, or combinations and subcombinations thereof, of FXYD5.

In some embodiments, antibodies may be screened against known related polypeptides to isolate an antibody population that specifically binds to FXYD5 polypeptides. For example, antibodies specific to human FXYD5 polypeptides will flow through a column comprising FXYD proteins (with the exception of FXYDS) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current Protocols in Immunology, Cooligan et aL (eds.), National Institutes of Health, John Wiley and Sons, Inc., 1995). Screening and isolation of specific antibodies is well known in the art (see, Fundamental Immunology, Paul (eds.), Raven Press, 1993; Gethoff et al., Adv. in Immunol. 43: 1-98, 1988; Monoclonal Antibodies: Principles and Practice, Goding, J. W. (eds.), Academic Press Ltd., 1996; Benjamin et aL, Ann. Rev. Immunol. 2: 67-101, 1984).

Representative examples of such assays include: concurrent immunoelectrophoresis, radioimmunoassay (RIA), radioimmunoprecipitation, enzyme-linked immimoscabent assay (ELISA), dot blot or Western blot assay, inhibition or competition assay, and sandwich assay.

In some embodiments, the antibodies of the present invention do not specifically bind to epitopes of FXYD5 selected from the group consisting of Thr19-A1a33 (SEQ ID NO:3), Leu54-Asp82 (SEQ ID NO:4), Pro89-A1a97 (SEQ ID NO:5), Pro100-Lys125 (SEQ ID NO:6), Ser127-Phe135 (SEQ ID NO:7). In some embodiments, the antibodies bind to an epitope of FXYD5 other than an epitope bound by mAbs NCC-3G10 or NCC-M53, described in Shimamura et al., J. Clin. Oncol., 21:659, 2003.

The invention also provides antibodies that are SMIPs or binding domain immunoglobulin fhsion proteins specific for target protein. These constructs include single-chain polypeptides comprising antigen binding domains fused to immunoglobulin domains necessary to carry out antibody effector functions. See e.g., WO03/041600, U.S. Patent publication 20030133939 and US Patent Publication 20030118592.

In some embodiments the antibodies of the present invention are neutralizing antibodies. In some embodiments the antibodies are targeting antibodies. In some embodiments, the antibodies are internalized upon binding a target. In some embodiments the antibodies do not become internalized upon binding a target and instead remain on the surface.

The antibodies of the present invention can be screened for the ability to either be rapidly internalized upon binding to the tumor-cell antigen in question, or for the ability to remain on the cell surface following binding. In some embodiments, for example in the construction of some types of immunoconjugates, the ability of an antibody to be internalized may be desired if internalization is required to release the toxin moiety. Alternatively, if the antibody is being used to promote ADCC or CDC, it may be more desirable for the antibody to remain on the cell surface. A screening method can be used to differentiate these type behaviors. For example, a tumor cell antigen bearing cell may be used where the cells are incubated with human IgG1 (control antibody) or one of the antibodies of the invention at a concentration of approximately 1 μg/mL on ice (with 0.1% sodium azide to block internalization) or 37° C. (without sodium azide) for 3 hours. The cells are then washed with cold staining buffer (PBS+1% BSA+40.1% sodium azide), and are stained with goat anti-human IgG-FITC for 30 minutes on ice. Geometric mean fluorescent intensity (MFI) is recorded by PACS Calibur. If no difference in MFI is observed between cells incubated with the antibody of the invention on ice in the presence of sodium azide and cells observed at 37° C. in the absence of sodium azide, the antibody will be suspected to be one that remains bound to the cell surface, rather than being internalized. If however, a decrease in surface stainable antibody is found when the cells are incubated at 37° C. in the absence of sodium azide, the antibody will be suspected to be one which is capable of internalization.

Antibody Conjugates

In some embodiments, the antibodies of the invention are conjugated. In some embodiments, the conjugated antibodies are useful for cancer therapeutics, cancer diagnosis, or imaging of cancerous cells.

For diagnostic applications, the antibody typically will be labeled with a detectable moiety. Numerous labels are available which can be generally grouped into the following categories:

(a) Radionuclides such as those discussed infra. The antibody can be labeled, for example, with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dilydroplithelazinedicmes, malate dehydrogenase, urease, peroxidase such as horseradish peroxidase (HRPO), alkaline phosphatase, .beta.-galactosidase, glucoamylase, lysozyme, saccharide oxidases (e.g., glucose oxides; galactose oxidase, and glucose-6-phosphate dehydrogenase), heterocyclic oxidases (such as unease and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

The antibodies may also be used for in vivo diagnostic assays. In some embodiments, the antibody is labeled with a radionuclide so that the tumor can be localized using immunoscintiography. As a matter of convenience, the antibodies of the present invention can be provided in a kit, i.e., a packaged combination of reagents in predetermined amounts with instructions for performing the diagnostic assay. Where the antibody is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In some embodiments, antibodies are conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody immtmocenjugate. In some embodiments, the conjugate may be the highly potent maytansine derivative DM1 (N2′-deacetyl-N2′-(3-mercapto-1-oxopropyl)-maytansine) (see for example WO02/098883 published Dec. 12, 2002) which has an IC50 of approximately 10-11 M (review, see Payne (2003) Cancer Cell 3:207- 212) or DM4 (N2′-deacetyl-N2′(4-methyl-4-mercapto-1-oxopentyl)-maytansine) (see for example WO2004/103272 published Dec. 2, 2004).

In some embodiments the antibody conjugate comprises an anti-tumor cell antigen antibody conjugated to one or more calicheamicin molecules. The calicheamicin family of antibiotics is capable of producing double-stranded DNA breaks at sub-picomolar concentrations. Structural analogues of calicheamicin which may be used include, but are not limited to, gamma1I, alpha2I, alpha3I, N-acetyl-gamma1I, PSAG and thetall (Hinman et al. Cancer Research 53: 3336-3342 (1993) and Lode et al., Cancer Research 58: 2925-2928 (1998)). See, also, U.S. Pat. Nos. 5,714,586; 5,712,374; 5,264,586; and 5,773,001, each of which is expressly incorporated herein by reference.

In some embodiments the antibody is conjugated to a prodrug capable of being release in its active form by enzymes overproduced in many cancers. For example, antibody conjugates can be made with a prodrug form of doxorubicin wherein the active component is released from the conjugate by plasmin. Plasmin is known to be over produced in many cancerous tissues (see Decy at al, (2004) FASEB Journal 18(3): 565-567).

In some embodiments the antibodies are conjugated to enzymatically active toxins and fragments thereof In some embodiments the toxins include, without limitation, diphtheria A chain, nonbinding active fragments of diphtheria toxin, exotoxin A chain (from Pseudomonas aeruginosa), Pseudomonas endotoxin, ricin A chain, abrin A chain, modeccin A chain, alpha-sarcin, Aleurites fordii proteins, dianthin proteins, Phytolaca americana proteins (PAPI, PAPII, and PAP-S), Ribonuclease (RNase), Deoxyribonuclease (Dnase), pokeweed antiviral protein, momordica charantia inhibitor, curcin, crotin, sapaonaria officinalis inhibitor, gelonin, mitogellin, restrictocin, phenomycin, neomycin and the tricothecenes. See, for example, WO 93/21232 published Oct. 28, 1993. In some embodiments the toxins have low intrinsic immunogenicity and a mechanism of action (e.g. a cytotoxic mechanism versus a cytostatic mechanism) that reduces the opportunity for the cancerous cells to become resistant to the toxin.

In some embodiments conjugates are made between the antibodies of the invention and immunomodulators. For example, in some embodiments immunostimulatory oligonucleotides can be used. These molecules are potent immunogens that can elicit antigen-specific antibody responses (see Datta at al, (2003) Ann N.Y. Acad. Sci 1002: 105-111). Additional immunomodulatory compounds can include stem cell growth factor such as “S1 factor”, lymphotoxins such as tumor necrosis factor (TNF), hematopoietic factor such as an interleukin, colony stimulating factor (CSF) such as granulocyte-colony stimulating factor (G-CSF) or granulocyte macrophage-stimulating factor (GM-CSF), interferon (IFN) such as interferon alpha, beta or gamma, erythropoietin, and thrombopoietin.

In some embodiments radioconjugated antibodies are provided. In some embodiments such antibodies can be made using ³²P, ³³P, ⁴⁷SC, ⁵⁹ Fe, ⁶⁴Cu, ⁶⁷Cu, ⁷⁵Se, ⁷⁷As, ⁸⁹Sr, ⁹⁰Y, 99Mo, ¹⁰⁵Rh, 109Pd, ¹²⁵I, ¹³¹I, ¹⁴²Pr, ¹⁴³Pr, ¹⁴⁹Pm, ¹⁵³Sm, ¹⁶¹Th, ¹⁶⁶Ho, ¹⁶⁹Er, ¹⁷⁷Lu, ¹⁸⁶Re, ¹⁸⁸Re, ¹⁸⁹Re, ¹⁹⁴Ir, ₁₉₈Au, ¹⁹⁹Au, ²¹¹Pb, ²¹²pb, ²¹³Bi, ⁵⁸Co, ⁶⁷Ga, ^{80m}Br, ^{99m}Tc, ^{103m}Rh, ¹⁰⁹Pt, 161Ho, ^{189m}Os, ¹⁹²Ir, ¹⁵²Dy, ²¹¹At, ²¹²Bi, ²²³Ra, ²¹⁹Rn, ²¹⁵Po, ²¹¹Bi, ²²⁵Ac, ²²¹Fr, ²¹⁷At, ²¹³Bi, ²⁵⁵Fm, and combinations and subcombinations thereof. In sonic embodiments, boron, gadolinium or uranium atoms are conjugated to the antibodies. In some embodiments the boron atom is ¹⁰B, the gadolinium atom is ¹⁵⁷Gd and the uranium atom is ²³⁵U.

In some embodiments the radionuclide conjugate has a radionuclide with an energy between 20 and 10,000 keV. The radionuclide can be an Auger emitter, with an energy of less than 1000 keV, a P emitter with an energy between 20 and 5000 keV, or an alpha or ‘α’ emitter with an energy between 2000 and 10,000 keV.

In some embodiments diagnostic radioconjugates are provided which comprise a radionuclide that is a gamma-, beta-, or positron-emitting isotope. In some embodiments the radionuclide has an energy between 20 and 10,000 keV. In some embodiments the radionuclide is selected from the group of ¹⁸F, ⁵¹Mn, ^{52m}Mn, ⁵²Fe, ⁵⁵Co, ⁶²Cu, ⁶⁴Cu, ⁶⁸Ga, ⁷²As, ⁷⁵Br, ⁷⁶Br, ^{82m}Rb, ⁸³Sr, ⁸⁹Zr, ^{94m}Tc, ⁵¹Cr, ⁵⁷Co, ⁵⁸Co, ⁵⁹Fe, ⁶⁷Ga, ⁷⁵Se, ⁹⁷Ru, ^{99m}Tc, ^{114m}In, ¹⁷⁴ ¹²³I, ¹²⁵I, ¹³Li and ¹⁹⁷Hg.

In some embodiments the antibodies of the invention are conjugated to diagnostic agents that are photoactive or contrast agents. Photoactive compounds can comprise compounds such as chromagens or dyes. Contrast agents may be, for example a paramagnetic ion, wherein the ion comprises a metal selected from the group of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). The contrast agent may also be a radio-opaque compound used in X-ray techniques or computed tomography, such as an iodine, iridium, barium, gallium and thallium compound. Radio-opaque compounds may be selected from the group of barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosularnide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride. In some embodiments, the diagnostic immanoconjugates may contain ultrasound-enhancing agents such as a gas filled liposome that is conjugated to an antibody of the invention. Diagnostic immunoconjugates may be used for a variety of procedures including, but not limited to, intraoperative, endoscopic or intravascular methods of tumor or cancer diagnosis and detection.

In some embodiments antibody conjugates are made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as his (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-diflunro-2,4-dinitrobenzene). For example, a ricin immunotoxin can be prepared as described in Vitetta et al. Science 238: 1098 (1987). Carbon-14-labeled 1-isothiocyanatobenzy1-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Cheri et al. Cancer Research 52: 127-131 (1992)) may be used. Agents may be additionally be linked to the antibodies of the invention through a carbohydrate moiety.

In some embodiments fusion proteins comprising the antibodies of the invention and cytotoxic agents may be made, e.g. by recombinant techniques or peptide synthesis. In some embodiments such immunoconjugates comprising the anti-tumor antigen antibody conjugated with a cytotoxic agent are administered to the patient. In some embodiments the immunoconjugate and/or tumor cell antigen protein to which it is bound is/are internalized by the cell, resulting in increased therapeutic efficacy of the immunoconjugate in killing the cancer cell to which it binds. In some embodiments, the cytotoxic agent targets or interferes with nucleic acid in the cancer cell. Examples of such cytotoxic agents include maytansinoids, calicheamicins, ribonucleases and DNA endonucleases.

In some embodiments the antibodies are conjugated to a “receptor” (such as streptavidin) for utilization in tumor pit-targeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

In some embodiments the antibodies are conjugated to a cytotoxic molecule which is released inside a target cell lysozome. For example, the drug monomethyl auristatin E (MMAE) can be conjugated via a valine-citrulline linkage which will be cleaved by the proteolytic lysozomal enzyme cathepsin B following internalization of the antibody conjugate (see for example WO03/026577 published Apr. 3, 2003). In some embodiments, the MMAE can be attached to the antibody using an acid-labile linker containing a hydrazone functionality as the cleavable moiety (see for example WO02/088172 published Nov. 11, 2002).

Antibody Dependent Enzyme Mediated Prodrug Therapy (ADEPT)

In some embodiments the antibodies of the present invention may be used in ADEPT by conjugating the antibody to a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

In some embodiments the enzyme component of the immunoconjugate useful for ADEPT includes any enzyme capable of acting on a prodrug in such a way so as to covert it into its more active, cytotoxic form.

Enzymes that are useful in ADEPT include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drags; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. In some embodiments antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

In some embodiments the ADEPT enzymes can be covalently bound to the antibodies by techniques well known in the art such as the use of the heterobifunctional crosslinking reagents discussed above. In some embodiments, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

In some embodiments identification of an antibody that acts in a cytostatic manner rather than a cytotoxic manner can be accomplished by measuring viability of a treated target cell culture in comparison with a non-treated control culture. Viability can be detected using methods known in the art such as the CellTiter-Blue® Cell Viability Assay or the CellTiter-Glo® Luminescent Cell Viability Assay (Promega, catalog numbers G8080 and G5750 respectively). In some embodiments an antibody is considered as potentially cytostatic if treatment causes a decrease in cell number in comparison to the control culture without any evidence of cell death as measured, for example, by the means described above.

In some embodiments an in vitro screening assay can be performed to identify an antibody that promotes ADCC using assays known in the art. One exemplary assay is the In Vitro ADCC Assay. To prepare chromium 51-labeled target cells, tumor cell lines are grown in tissue culture plates and harvested using sterile 10 mM EDTA in PBS. The detached cells are washed twice with cell culture medium. Cells (5×10⁶) are labeled with 200 pCi of chromium 51 (New England Nuclear/DuPont) at 37° C. for one hour with occasional mixing. Labeled cells were washed three times with cell culture medium, then are resuspended to a concentration of 1×10⁵ cells/mL. Cells are used either without opsonization, or are opsonized prior to the assay by incubation with test antibody at 100 ng/mL and 1.25 ng/mL in PBMC assay or 20 ng/mL and 1 ng/mL in NK assay. Peripheral blood mononuclear cells are prepared by collecting blood on heparin from normal healthy donors and diluted with an equal volume of phosphate buffered saline (PBS). The blood is then layered over LYMPHOCYTE SEPARATION MEDIUM® (LSM: Organon Telaulca) and centrifuged according to the manufacturer's instructions. Mononuclear cells are collected from the LSM-plasma interface and are washed three times with PBS. Effector cells are suspended in cell culture medium to a final concentration of 1×10⁷ cells/mL. After purification through LSM, natural killer (NK) cells are isolated from PBMCs by negative selection using an NK cell isolation kit and a magnetic column (Miltenyi Biotech) according to the manufacturer's instructions. Isolated NK cells are collected, washed and resuspended in cell culture medium to a concentration of 2×10⁶ cells/mL. The identity of the NK cells is confirmed by flow cytometric analysis. Varying effector: target ratios are prepared by serially diluting the effector (either PBMC or NK) cells two-fold along the rows of a microtiter plate (100 μL final volume) in cell culture medium. The concentration of effector cells ranges from 1.0×10⁷/mL to 2.0×10⁴/mL for PBMC and from 2.0×10⁶/mL to 3.9×10³/mL for NK. After titration of effector cells, 100 μL of chromium 51-labeled target cells (opsonized or nonoponsonized) at 1×10⁵ cells/mL are added to each well of the plate. This results in an initial effectortarget ratio of 100:1 for PBMC and 20:1 for NK cells. All assays are run in duplicate, and each plate contains controls for both spontaneous lysis (no effector cells) and total lysis (target cells plus 100 μL 1% sodium dodecyl sulfate, 1 N sodium hydroxide). The plates are incubated at 37° C. for 18 hours, after which the cell culture supernatants are harvested using a supernatant collection system (Skatron Instrument, Inc.) and counted in a Minaxi auto-gamma 5000 series gamma counter (Packard) for one minute. Results are then expressed as percent cytotoxicity using the formula: % Cytotoxicity=(sample cpm-spontaneous lysis)/(total lysis-spontaneous lysis)×100.

To identify an antibody that promotes CDC, the skilled artisan may perform an assay known in the att. One exemplary assay is the In Vitro CDC assay. In vitro, CDC activity can be measured by incubating tumor cell antigen expressing cells with human (or alternate source) complement-containing serum in the absence or presence of different concentrations of test antibody. Cytotoxicity is then measured by quantifying live cells using ALAMAR BLUE® (Gazzano-Santoro et al., J. hnmunol. Methods 202 163-171 (1997)). Control assays are performed without antibody, and with antibody, but using heat inactivated serum and/or using cells which do not express the tumor cell antigen in question. Alternatively, red blood cells can be coated with tumor antigen or peptides derived from tumor antigen, and then CDC may be assayed by observing red cell lysis (see for example Karjalainen and Mantyjarvi, Acta Pathol Microbiol Scand. 1981 Oct 89(5):315-9).

To select for antibodies that induce cell death, loss of membrane integrity as indicated by, e.g., propidium iodide (PI), trypan blue or 7-aminoactinomycin D (7AAD) uptake may be assessed relative to control. One exemplar/assay is the PI uptake assay using tumor antigen expressing cells. According to this assay, tumor cell antigen expressing cells are cultured in Dulbecco's Modified Eagle Medium (D-MEM):Ham's F-12 (50:50) supplemented with 10% heat-inactivated FBS (Hyclone) and 2 mM L-glutamine. (Thus, the assay is performed in the absence of complement and immune effector cells). The tumor cells are seeded at a density of 3×10⁶ per dish in 100×20 mm dishes and allowed to attach overnight The medium is then removed and replaced with fresh medium alone or medium containing 10 μg/mL of the appropriate monoclonal antibody. The cells are incubated for a 3 day time period. Following each treatment, monolayers are washed with PBS and detached by trypsinization. Cells are then centrifuged at 1200 rpm for 5 minutes at 4° C., the pellet resuspended in 3 mL ice cold Ca²⁺ binding buffer (10 mM Hepes, pH 7.4, 140 mM NaCl, 2.5 mM CaCl₂) and aliquoted into 35 mm strainer-capped 12×75 tubes (1 mL per tube, 3 tubes per treatment group) for removal of cell clumps. Tubes then receive PI (10 μg/mL). Samples may be analyzed using a FACSCAN™ flow cytometer and FACSCONVERT™. CellQuest software (Becton Dickinson). Those antibodies that induce statistically significant levels of cell death as determined by PI uptake may be selected as cell death-inducing antibodies.

Antibodies can also be screened in vivo for apoptotic activity using ¹⁸F-annexin as a PET imaging agent In this procedure, Annexin V is radiolabeled with ¹⁸F and given to the test animal following dosage with the antibody under investigation. One of the earliest events to occur in the apoptotic process is the eversion of phosphatidylserine from the inner side of the cell membrane to the outer cell surface, where it is accessible to annexin. The animals are then subjected to PET imaging (see Yagle et al., J Nucl Med. 2005 April 46(4):658-66). Animals can also be sacrificed and individual organs or tumors removed and analyzed for apoptotic markers following standard protocols.

While in some embodiments cancer may be characterized by overexpression of a gene expression product, the present application further provides methods for treating cancer which is not considered to be a tumor antigen-overexpressing cancer. To determine tumor antigen expression in the cancer, various diagnostic/prognostic assays are available. In some embodiments, gene expression product overexpression can be analyzed by immunohistochemistry (IHC). Paraffin embedded tissue sections from a tumor biopsy may be subjected to the IHC assay and accorded a tumor antigen protein staining intensity criteria as follows:

Score 0: no staining is observed or membrane staining is observed in less than 10% of tumor cells.

Score 1+: a faint/barely perceptible membrane staining is detected in more than 10% of the tumor cells. The cells are only stained in part of their membrane.

Score 2+: a weak to moderate complete membrane staining is observed in more than 10% of the tumor cells.

Score 3+: a moderate to strong complete membrane staining is observed in more than 10% of the tumor cells.

Those tumors with 0 or 1+ scores for tumor antigen overexpression assessment may be characterized as not overexpressing the tumor antigen, whereas those tumors with 2+ or 3+ scores may be characterized as overexpressing the tumor antigen.

Alternatively, or additionally, fluorescence In situ hybridization (FISH) assays such as the INFORM™, (sold by Ventana, Ariz) or PATHVISIONT™ (Vysis, Ill.) may be carried out on formalin-fixed, paraffin-embedded tumor tissue to determine the extent (if any) of tumor antigen overexpression in the tumor.

Additionally, antibodies can be chemically modified by covalent conjugation to a polymer to increase their circulating half-life, for example. Each antibody molecule may be attached to one or more (i.e. 1, 2, 3, 4, 5 or more) polymer molecules. Polymer molecules are preferably attached to antibodies by linker molecules. The polymer may, in general, be a synthetic or naturally occurring polymer, for example an optionally substituted straight or branched chain polyalkene, polyalkenylene or polyoxyalkylene polymer or a branched or unbranched polysaccharide, e.g. homo- or hetero-polysaccharide. In some embodiments the polymers are polyoxyethylene polyols and polyethylene glycol (PEG). PEG is soluble in water at room temperature and has the general formula: R(O—CH₂—CH₂)ₙ O—R where R can be hydrogen, or a protective group such as an alkyl or alkanol group. In some embodiments, the protective group has between 1 and 8 carbons. In some embodiments the protective group is methyl. The symbol n is a positive integer, between 1 and 1,000, or 2 and 500. In some embodiments the PEG has an average molecular weight between 1000 and 40,000, between 2000 and 20,000, or between 3,000 and 12, 000. In some embodiments, PEG has at least one hydroxy group. In some embodiments the hydroxy is a terminal hydroxy group. In some embodiments it is this hydroxy group which is activated to react with a free amino group on the inhibitor. However, it will be understood that the type and amount of the reactive groups may be varied to achieve a covalently conjugated PEG/antibody of the present invention. Polymers, and methods to attach them to peptides, are shown in U.S. Pat. Nos. 4,766,106; 4,179,337; 4,495,285; and 4,609,546, each of which is hereby incorporated by reference in its entirety.

Safety Studies

The antibodies of the invention can be examined for safety and toxicological characteristics. Guidelines for these types of studies can be found in the document issued by the USDA CBER division, “Points to Consider in the Manufacture and Testing of Monoclonal Antibody Products for Human Use” (Docket No. 94D-0259, Feb. 28, 1997) incorporated herein by reference. In general, the candidate antibodies should be screened in preclinical studies using a number of human tissue samples and/or isolated human cell types to assess non-target tissue binding and cross reactivity. Following a satisfactory outcome from these human tissue studies, a panel of tissue samples or isolated cells from a variety of animal species can be screened to identify a suitable species for use in general toxicological studies. If no cross reactive animal species is identified, other types of models may be deemed appropriate. These other models can include studies such as xenograft models, where human tumor cells are implanted into a rodent host, or the use of a surrogate monoclonal antibody which recognizes the corresponding tumor-cell antigen in the animal species chosen for the toxicological studies. It should be appreciated that the data from these types of alternate models will be first approximations and proceeding into higher species should be done with caution.

For a candidate naked antibody, studies looking at simple tolerability can be performed. In these studies the therapeutic index of the candidate molecule can be characterized by observing any dose-dependent pharmacodynamic effects. A broad range of doses may be used (for example from 0.1 mg/kg to 100 mg/kg). Differences between tumor cell antigen number, affinity of the candidate antibody for the cross-reactive animal target and differences in cellular response following binding of the antibody should be considered in estimating therapeutic index. Pharmacodynamic and pharmacokinetic studies should also be carried out in an appropriate animal model to help guild initial dose considerations when the candidate antibody is tested in humans.

For candidate immunoconjugaWs, stability studies of the conjugate must be performed in vivo. Optimally, pharmacodynamic and pharmacokinetic studies should be carried out on the individual components of the immunoconjugate to determine the consequences of any breakdown products from the candidate immunoconjugate. Pharmacodynamic and pharmacokinetic studies should also be carried out as above in an appropriate animal model to help guild initial dose considerations. Additional consideration must be given to safety study design when the drug will be given in combination with pretreatment with naked antibody. Safety studies must be carried out with the naked antibody alone, and studies must be designed with the immunoconjugate keeping in mind that the ultimate doses of immunoconjugate will be lower in this type of treatment regimen.

For radio-immunoconjugates, animal tissue distribution studies should be carried out to determine biodistribution data. In addition, an accounting of metabolic degradation of the total dose of administered radioactivity should be performed with both early and late time points being taken. Radio-immunoconjugates can be tested for stability in vitro using serum or plasma, and methods should be developed to measure the percentages of free radionuclide, radio-immunoconjugate and labeled, non-antibody compounds.

Oligonucleotides

In some embodiments, the FXYD5 modulator is an oligonucleotide. In some embodiments, the FXYD5 modulator is an oligonucleotide comprising a sequence selected from the group consisting of SEQ ID NOs:12-26

In some embodiments the oligonucleotide is an antisense or RNAi oligonucleotide. In some embodiments the oligonucleotide is complementary to a region, domain, portion, or segment of the FXYDS gene or gene expression product. In some embodiments, the oligonucleotide comprises from about 5 to about 100 nucleotides, from about 10 to about 50 nucleotides, from about 12 to about 35, and from about 18 to about 25 nucleotides. In some embodiments, the oligonucleotide is at least 50%, at least 75%, at least 80%, at least 90%, at least 95%, at least 96%, at least 97%, at least 98%, at least 99%, or 100% homologous to a region, portion, domain, or segment of the FXYD5 gene or gene expression product In some embodiments there is substantial sequence homology over at least 15, 20, 25, 30, 35, 40, 50, or 100 consecutive nucleotides of the FXYD5 gene or gene product. In some embodiments there is substantial sequence homology over the entire length of the FXYD5 gene or gene expression product In some embodiments, the oligonucleotide binds under moderate or stringent hybridization conditions to a nucleic acid molecule having a rurcleotide sequence of SEQ ID NO:1.

In some embodiments, the FXYD5 modulator is a double stranded RNA (dsRNA) molecule and works via RNAi (RNA interference). In some embodiments, one strand of the dsRNA is at least 50%, at least 75%, at least 80%, at least 90%, at least 95%, at least 96%, at least 97%, at least 98%, at least 99%, or 100% homologous to a region, portion, domain, or segment of the FXYD5 gene. In some embodiments there is substantial sequence homology over at least 15, 20, 25, 30, 35, 40, 50, 100, 200, 300, 400, 500, or 1000 consecutive nucleotides of the FXYD5 gene. In some embodiments there is substantial or complete sequence homology over the entire length of the FXYD5 nucleotide sequence, or its complement.

In some embodiments oligonucleotides of the invention are used in a polymerase chain reaction (PCR). This sequence may be based on (or designed from) a genomic sequence or cDNA sequence and is used to amplify, confirm, or detect the presence of an identical, similar, or complementary DNA or RNA in a particular cell or tissue.

Small Molecules

In some embodiments, the FXYD5 modulator is a small molecule. As used herein, the teen “small molecule” refers to an organic or inorganic non-polymer compound that has a molecular weight that is less than about 10 kilodaltons. Examples of small molecules include peptides, oligonucleotides, organic compounds, inorganic compounds, and the like. In some embodiments, the small molecule has a molecular weight that is less than about 9, about 8, about 7, about 6, about 5, about 4, about 3, about 2, or about 1 kilodalton.

Mimetics

In some embodiments, the FXYD5 modulator is a mimetic. As used herein, the term “mimetic” is used to refer to compounds which mimic the activity of a peptide. Mimetics are non-peptides but may comprise amino acids linked by non-peptide bonds. U.S. Pat. No. 5,637,677, issued on Jun. 10, 1997, and parent applications thereof; all of which are incorporated herein by reference, contain detailed guidance on the production of mimetics. Briefly, the three-dimensional structure of the peptides which specifically interacts with the three dimensional structure of the FXYD5 is duplicated by a molecule that is not a peptide. In some embodiments the FXYD5 mimetic is a mimetic of FXYD5 or a mimetic of a ligand of FXYD5.

Decoys

In some embodiments, the FXYD5 modulator is a decoy comprising at least a portion of a FXYD5 polypeptide. In some embodiments the decoy competes with natural FXYD5 polypeptides for binding to an FXYD5 ligand. In some embodiments, the decoy is labeled to facilitate quantification, qualification, and/or visualization. In other embodiments, the decoy further comprises a moiety to facilitate isolation and/or separation of the decoy or the decoy-ligand complex. In some embodiments the decoy comprises at least a portion of a FXYD5 polypeptide fused to an antibody or antibody fragment.

Methods of Treating/Preventing Cancer

The present invention provides methods for treating and/or preventing cancer or symptoms of cancer in a subject comprising administering to the subject a therapeutically effective amount of one or more FXYD5 modulators of the present invention. In some embodiments the cancer is a cancer associated with overexpression of FXYD5. In some embodiments, the cancer is colon cancer, breast cancer, skin cancer, esophageal cancer, liver cancer, pancreatic cancer, prostatic cancer, uterine cancer, cervical cancer, lung cancer, bladder cancer, ovarian cancer, multiple myeloma or melanoma. In some embodiments, the cancer is in a non-hormonally regulated tissue. In some embodiments the breast cancer is an ER-positive breast cancer, an ER-negative breast cancer, or a metastatic breast cancer. In some embodiments the breast cancer is ductal adenocarcinoma, lobular adenocarcinoma, or metastatic adenocarcinoma. In some embodiments the subject has been diagnosed as having a cancer or as being predisposed to cancer.

Symptoms of cancer are well-known to those of skill in the art and include, without limitation, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, hmg metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreas metastases, difficulty swallowing, and the like.

A therapeutically effective amount of the modulating compound can be determined empirically, according to procedures well known to medicinal chemists, and will depend, inter alia, on the age of the patient, severity of the condition, and on the ultimate pharmaceutical formulation desired. Administration of the modulators of the present invention can be carried out, for example, by inhalation or suppository or to mucosal tissue such as by lavage to vaginal, rectal, urethral, buccal and sublingual tissue, orally, topically, intranasally, intraperitoneally, parenterally, intravenously, intraiymphatically, intratumorly, intramuscularly, interstitially, intra-arterially, subcutaneously, intraoccularly, intrasynovial, transepithelial, and transderrnally. In some embodiments, the inhibitors are administered by lavage, orally or inter-arterially. Other suitable methods of introduction can also include rechargeable or biodegradable devices and slow or sustained release polymeric devices. As discussed above, the therapeutic compositions of this invention can also be administered as part of a combinatorial therapy with other known anti-cancer agents or other known anti-bone disease treatment regimen.

The present invention further provides methods of modulating a FXYD5-related biological activity in a patient. The methods comprise administering to the patient an amount of a FXYD5 modulator effective to modulate one or more FXYD5 biological activities. Suitable assays for measuring FXYD5 biological activities are set forth supra and infra.

The present invention also provides methods of inhibiting cancer cell growth in a patient in need thereof comprising administering a therapeutically effective amount of one or more FXYD5 modulators to the patient Suitable assays for measuring FXYD5-related cell growth are known to those skilled in the art and are set forth supra and infra.

The present invention further provides methods of inhibiting cancer in a patient in need thereof. The methods comprise determining if the patient is a candidate for FXYD5 therapy as described herein and administering a therapeutically effective amount of one or more FXYD5 modulators to the patient if the patient is a candidate for FXYD5 therapy. If the patient is not a candidate for FXYD5 therapy, the patient is treated with conventional cancer treatment.

The present invention provides methods of inhibiting cancer in a patient diagnosed or suspected of having a cancer. The methods comprise administering a therapeutically effective amount of one or more FXYD5 modulators to the patient.

The present invention also provides methods of inducing apoptosis in a population of cells expressing FXYD5. In some embodiments the methods comprise contacting the population of cells with a FXYD5 modulator in conjunction with a chemotherapeutic agent In some embodiments, the FXYD5 modulator inhibits FXYD5 protein levels. In some embodiments, contacting the population of cells with the FXYD5 modulator in conjunction with a chemotherapeutic has an additive effect.

The present invention also provides methods for inhibiting the interaction of two or more cells in a patient comprising administering a therapeutically effective amount of a FXYD5 modulator to said patient Suitable assays for measuring FXYD5-related cell interactions are known to those skilled in the art and are set forth supra and infra.

The present invention also provides methods of modulating one or more symptoms of cancer in a patient comprising administering to said patient a therapeutically effective amount of the FXYD5 compositions described herein.

The present invention further provides methods for inhibiting cell growth in a patient in need thereof comprising administering to the patient a therapeutically effective amount of a FXYD5 modulator. Suitable assays for measuring FXYD5-related anchorage-independent cell growth are set forth supra and infra.

The present invention also provides methods for inhibiting migration of cancer cells in a patient in need thereof comprising administering to the patient a therapeutically effective amount of a FXYD5 modulator. Suitable assays for measuring FXYD5-related cell migration are known to those skilled in the art.

The present invention further provides methods for inhibiting adhesion of cancer cells in a patient in need thereof comprising administering to the patient a therapeutically effective amount of a FXYD5 modulator. Suitable assays for measuring FXYD5-related cell adhesion are known to those dulled in the art.

The present invention also provides methods to prophylactically treat a patient who is predisposed to develop cancer, a cancer metastasis or who has had a metastasis and is therefore susceptible to a relapse or recurrence. The methods are particularly useful in high-risk individuals who, for example, have a foully history of cancer or of metastasizing tumors, or show a genetic predisposition for a cancer metastasis. In some embodiments the tumors are FXYD5-related tumors. Additionally, the methods are useful to prevent patients from having recurrences of FXYD5-related tumors who have had FXYD5-related tumors removed by surgical resection or treated with a conventional cancer treatment.

The present invention also provides methods of inhibiting cancer progression and/or causing cancer regression comprising administering to the patient a therapeutically effective amount of a FXYD5 modulator.

In some embodiments, the patient in need of anti-cancer treatment is treated with the FXYD5 modulators of the present invention in conjunction with chemotherapy and/or radiation therapy. For example, following administration of the FXYD5 modulators, the patient may also be treated with a therapeutically effective amount of anti-cancer radiation. In some embodiments chemotherapeutic treatment (for example with methotrettate and/or doxorubicine) is provided in combination with FXYD5 modulators. In some embodiments FXYD5 modulators are administered in combination with chemotherapy and radiation therapy.

Methods of treatment comprise administering single or multiple doses of one or more FXYD5 modulators to the patient In some embodiments the FXYD5 modulators are administered as injectable pharmaceutical compositions that are sterile, pyrogen free and comprise the FXYD5 modulators in combination with a pharmaceutically acceptable carrier or diluent.

In some embodiments, the therapeutic regimens of the present invention are used with conventional treatment regimens for cancer including, without limitation, surgery, radiation therapy, hormone ablation and/or chemotherapy. Administration of the FXYD5 modulators of the present invention may take place prior to, simultaneously with, or after conventional cancer treatment. In some embodiments, two or more different FXYD5 modulators are administered to the patient.

In some embodiments the amount of FXYD5 modulator administered to the patient is effective to inhibit one or more of cancer cell growth, tumor formation, cancer cell proliferation, cancer cell metastasis, cell migration, angiogenesis, FXYD5 signaling, inhibit FXYD5-mediated cell-cell adhesion, FXYD5-mediated inhibition of cell-cell membrane interaction, FXYD5-mediated cell-extracellular matrix interaction, integrin mediated activities, FXYD5-mediated cell-extracellular matrix degradation, and FXYD5 expression. In some embodiments the amount of FXYD5 modulator administered to the patient is effective to increase cancer cell death through apoptosis.

Combination Therapy

In some embodiments the invention provides compositions comprising two or more FXYD5 modulators to provide still improved efficacy against cancer. In some embodiments the FXYD5 modulators are monoclonal antibodies. Compositions comprising two or more FXYD5 antibodies may be administered to persons or mammals suffering from, or predisposed to suffer from, cancer. One or more antibodies may also be administered with another therapeutic agent, such as a cytotoxic agent, or cancer chemotherapeutic. Concurrent administration of two or more therapeutic agents does not require that the agents be administered at the same time or by the same route, as long as there is an overlap in the time period during which the agents are exerting their therapeutic effect Simultaneous or sequential administration is contemplated, as is administration on different days or weeks.

In some embodiments the methods provide of the invention contemplate the administration of combinations, or “cocktails”, of different antibodies. Such antibody cocktails may have certain advantages inasmuch as they contain antibodies which exploit different effector mechanisms or combine directly cytotoxic antibodies with antibodies that rely on immune effector functionality. Such antibodies in combination may exhibit synergistic therapeutic effects.

In some embodiments, combination therapy provides enhanced treatment. By “enhanced treatment” is meant any additive, synergistic, or potentiating effect For example, in LNCaP, combining FXYD5 knockdown with chemotherapeutic has an additive effect. In some embodiments the enhanced treatment comprises administering one or more FXYD5 modulators in conjunction with one or more chemotherapeutic agents.

A cytotoxic agent refers to a substance that inhibits or prevents the function of cells and/or causes destruction of cells. The term is intended to include radioactive isotopes (e.g., ¹³¹I, ¹²⁵I, ⁹⁰Y and ¹⁸⁶Re, chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody according to the invention.

In some embodiments, conventional cancer medicaments are administered with the compositions of the present invention. Conventional cancer medicaments include:

a) cancer chemotherapeutic agents;

b) additional agents;

c) prodrugs.

Cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; foible acid; purine analog antimetabolites, mercaptopurine; pyrimidine analog antimetabolites, such as fluorouracil (5-FU) and gemcitabine (Gemzare); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, carminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimer10), interferon-β, mepitiostane, mitobronitol, melphalan, laminin peptides, lentinan, Coriolus versicolor extract, tegafar/uracil, estramustine (estrogen/mechlorethamine).

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (INF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Prodrug refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wllman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, b-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into. a prodrug form for use herein include, but are not limited to, those chemotherapeutic agents described above.

Clinical Aspects

In some embodiments, the methods and compositions of the present invention are particularly useful in colon cancer, breast cancer, skin cancer, esophageal cancer, liver cancer, pancreatic cancer, prostatic cancer, uterine cancer, cervical cancer, lung cancer, bladder cancer, ovarian cancer, multiple myeloma and melanoma. In some embodiments, the cancer is ductal adenocarcinoma, lobular adenocarcinoma, or metastatic adenocarcinoma.

Pharmaceutical Compositions

The present invention also provides pharmaceutical compositions comprising one or more of the FXYD5 modulators described herein and a pharmaceutically acceptable carrier. In some embodiments the pharmaceutical compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the hire. A thorough discussion of pharmaceutically acceptable excipients is available in Remington: The Science and Practice of Pharmacy (1995) Alfonso Gennaro, Lippincott, Williams, & Wilkins.

Methods of Detecting EXYDS

The present invention also provides methods for detecting FXYD5. In some embodiments the FXYD5 is present in a patient or in a patient sample. In some embodiments the method comprises administering a composition comprising one or more FXYD5 modulators to the patient and detecting the localization of the imaging agent in the patient In some embodiments the patient sample comprises cancer cells. In some embodiments the FXYD5 modulator is linked to an imaging agent or is detectably labeled. In some embodiments, the FXYD5 modulator is a FXYD5 antibody conjugated to an imaging agent and is administered to a patient to detect one or more tumors or to determine susceptibility of the patient to FXYD5 therapy. The labeled antibodies will bind to the high density of receptors on cells and thereby accumulate on the tumor cells. Using standard imaging techniques, the site of the tumors can be detected.

The present invention also provides methods of imaging/detecting cells or tumors expressing or overexpressing FXYD5 comprising contacting a composition comprising an FXYD5 modulator to a sample and detecting the presence of the FXYD5 modulator in the sample. In some embodiments the sample is a patient sample. In some embodiments the patient sample comprises cancer cells. In some embodiments the FXYD5 modulator is linked to an imaging agent or is detectably labeled.

The present invention also provides methods for quantifying the amount of FXYD5 present in a patient, cell or sample. The methods comprise administering one or more of antibodies, probes, or small molecules to a patient or sample and detecting the amount of FXYD5 present in the sample. In some embodiments the antibodies, probes, or small molecules are linked to an imaging agent or are detectably labeled. Such information indicates, for example, whether or not a tumor is related to FXYD5, and, therefore, whether specific treatments should be used or avoided. In some embodiments, using standard techniques well known to the art-skilled, samples believed to include tumor cells are obtained and contacted with labeled antibodies, probes, oligonucleotides, and small molecules. After removing any unbound, labeled antibodies, probes, oligonucleotides or small molecules, the quantity of labeled antibodies, peptides, oligonucleotides or mimetics bound to the cell, or the quantity of antibodies, peptides, oligonucleotides or mimetics removed as unbound is determined. The information directly relates to the amount of FXYD5 present

Imaging can be performed using procedures well known to those of ordinary skill in the art. Imaging can be performed, for example, by radioscintigraphy, nuclear magnetic resonance imaging (MRI) or computed tomography (CT scan). The most commonly employed radiolabels for imaging agents include radioactive iodine and indium. Imaging by CT scan may employ a heavy metal such as an iron chelate. MRI scanning may employ chelates of gadolinium or manganese. Additionally, positron emission tomography (PET) may be possible using positron emitters of oxygen, nitrogen, iron, carbon, or gallium. Such methods are known to those skilled in the art. Examples of such methods are discussed by A. Takeda et al, Cancer Research 61, 5065-5069, Jul. 1, 2001; and C. Frederickson, Sci STKE. 2003 May 13; 2003(182); each of which is incorporated by reference in its entirety.

In some embodiments the FXYD5 modulator is a FXYDS antibody. In some embodiments the modulator is linked to an imaging agent or is detectably labeled. In some embodiments the imaging agent is ¹⁸F, ⁴³K, ⁵²Fe, ⁵⁷Co, ⁶⁷Cu, ⁶⁷Ga, ⁷⁷Br, ⁸⁷MSr, ⁸⁶Y, ⁹⁰Y, ⁹⁹MTc, ¹¹¹In, ¹²³I, ¹²⁵I, ¹²⁷Cs, ¹²⁹Cs, ¹³¹I, ¹³²I, ¹⁹⁷Hg, ²⁰³Pb, or ²⁰⁶Bi.

Methods of detection are well known to those of skill in the art. For example, methods of detecting polynucleotides include, but are not limited to PCR, Northern blotting Southern blotting, RNA protection, and DNA hybridization (including in situ hybridization). Methods of detecting polypeptides include, but are not limited to, Western blotting, ELISA, enzyme activity assays, slot blotting, peptide mass fingerprinting, electrophoresis, immunochemistry and immunohistochemistry. Other examples of detection methods include, but are not limited to, radioimmunoassay (RIA), chemiluminescence immunoassay, fluoroimmunoassay, time-resolved fluoroimmunoassay (TR-FIA), two color fluorescent microscopy, or inunnnochromatographic assay (ICA), all well known by those of skill in the art. In some preferred embodiments of the present invention, polynucleotide expression is detected using PCR methodologies and polypeptide production is detected using ELISA technology.

Methods for Delivering a Cytotoxic Agent or a Diagnostic Agent to a Cell

The present invention also provides methods for delivering a cytotoxic agent or a diagnostic agent to one or more cells that express FXYD5. In some embodiments the methods comprise contacting a FXYD5 modulator of the present invention conjugated to a cytotoxic agent or diagnostic agent with the cell.

Methods for Determining the Prognosis of a Cancer Patient

In some embodiments, methods for determining the prognosis of a patient with a FXYD5 associated cancer comprise detecting FXYDS bound to the plasma membrane of a cell in a patient sample. In some embodiments, detection of FXYDS bound to the plasma membrane of a cell in a patient sample is not indicative of a good prognosis for extended survival and or successful treatment with a FXYD5 modulator of the present invention and/or a conventional cancer medicament.

In some embodiments FXYD5 is encoded for by a nucleic acid having a sequence of SEQ ID NO:1 . In some embodiments FXYD5 has a sequence of SEQ ID NO:2.

Methods for Determining Susceptibility to FXYD5 Therapy

The present invention also provides methods for determining the susceptibility of a patient to FXYD5 therapy. The methods comprise detecting the presence or absence of evidence of differential expression of FXYD5 in a patient or patient sample. The presence of evidence of differential expression of FXYD5 in the patient or sample is indicative of a patient who is susceptible to FXYD5 therapy. In some embodiments, the absence of evidence of differential expression of FXYD5 in the patient or patient sample is indicative of a patient who is not a candidate for FXYD5 therapy.

In some embodiments the therapeutic methods comprise first identifying patients susceptible to FXYD5 therapy comprising administering to the patient in need thereof a composition comprising a FXYD5 modulator linked to an imaging agent and detecting the presence or absence of evidence of the gene or gene product in the patient. In some embodiments, the therapeutic methods farther comprise administering one or more FXYD5 modulators to the patient if the patient is a candidate for FXYD5 therapy and treating the patient with conventional cancer treatment if the patient is not a candidate FXYD5 therapy.

In some therapeutic methods, one or more FXYD5 modulators are administered to the patients alone or in combination with other anti-cancer medicaments when the patient is identified as having a cancer or being susceptible to a cancer.

Methods for Assessing the Progression of Cancer

The invention also provides methods for assessing the progression of cancer in a patient comprising comparing the level of an expression product of FXYD5 in a biological sample at a first time point to a level of the same expression product at a second time point A change in the level of the expression product at the second time point relative to the first time point is indicative of the progression of the cancer.

Methods for Screening

The present invention also provides methods of screening for anti-cancer agents. The methods comprise contacting a cell expressing FXYD5 with a candidate compound and determining whether an FXYD5-related biological activity is modulated. In some embodiments, inhibition of one or more of cancer cell growth, integrin mediated activities, cadherin mediated activities, tumor formation, cancer cell proliferation, cancer cell metastasis, cell migration, angiogenesis, FXYD5 signaling, FXYD5-mediated inhibition of cell-cell adhesion, and FXYD5 expression is indicative of an anti-cancer agent.

The present invention further provides methods of identifying a cancer inhibitor. The methods comprise contacting a cell expressing FXYD5 with a candidate compound and an FXYD5 ligand, and determining whether an FXYD5-related biological activity is modulated. In some embodiments, inhibition of one or more of cancer cell growth, integrin mediated activities, cadherin mediated activities, tumor formation, cancer cell proliferation, cancer cell metastasis, cell migration, angiogenesis, FXYD5 signaling, FXYD5-mediated inhibition of cell-cell adhesion, and FXYDS expression is indicative of a cancer inhibitor. In some embodiments the amount of FXYD5 modulator administered to the patient is effective to increase cancer cell apoptosis.

In some embodiments, the invention provides methods of screening for anti-cancer agents, particularly anti-metastatic cancer agents, by, for example, screening putative modulators for an ability to modulate the activity or level of a downstream marker.

Methods for Purifying FXYDS

In some embodiments, the invention provides methods of purifying FXYD5 protein from a sample comprising FXYD5. The methods comprise providing an affinity matrix comprising a FXYD5 antibody of the present invention bound to a solid support, contacting the sample with the affinity matrix to form an affinity matrix-FXYD5 protein complex, separating the affinity matrix-FXYD5 protein complex from the remainder of the sample; and releasing FXYD5 protein from the affinity matrix.

Kits

In some embodiments, the present invention provides kits for imaging and/or detecting a gene or gene product correlated with FXYD5 overexpression. Kits of the invention comprise detectable antibodies, small molecules, oligonucleotides, decoys, mimetics or probes as well as instructions for performing the methods of the invention. Optionally, kits may also contain one or more of the following: controls (positive and/or negative), containers for controls, photographs or depictions of representative examples of positive and/or negative results.

Each of the patents, patent applications, accession numbers and publications described herein is hereby incorporated by reference in its entirety.

Various modifications of the invention, in addition to those described herein, will be apparent to those of skill in the art in view of the foregoing description. Such modifications are also intended to fall within the scope of the appended embodiments. The present invention is further demonstrated in the following examples that are for purposes of illustration and are not intended to limit the scope of the present invention.

### EXAMPLES

### Example 1

### FXYD5 Gene Expression Analyses

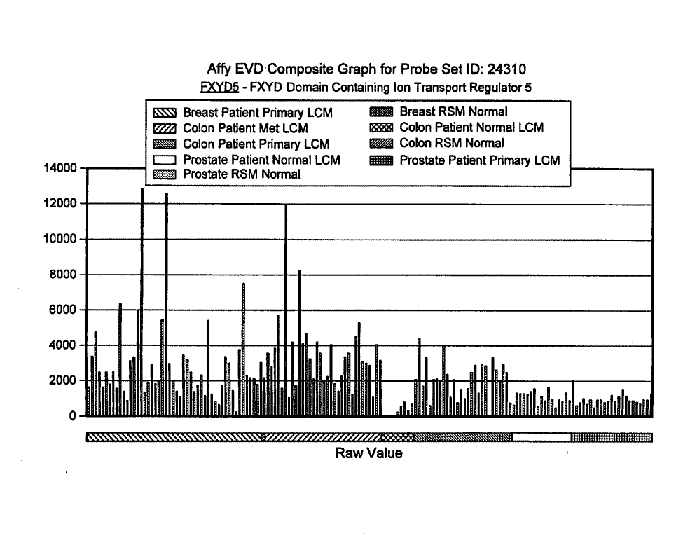

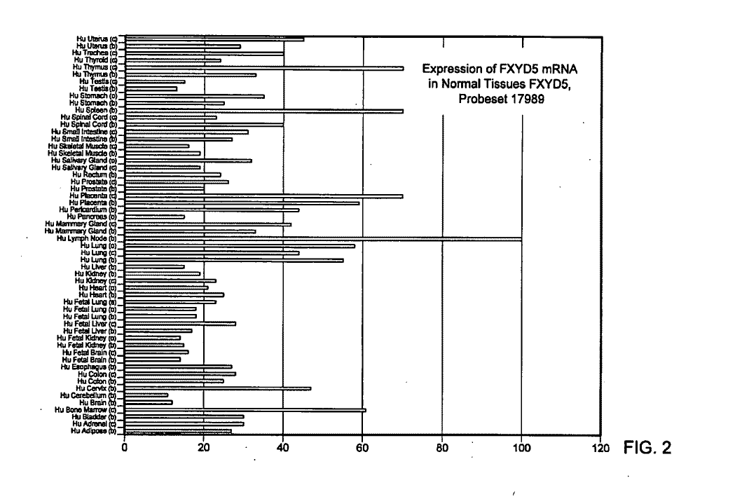

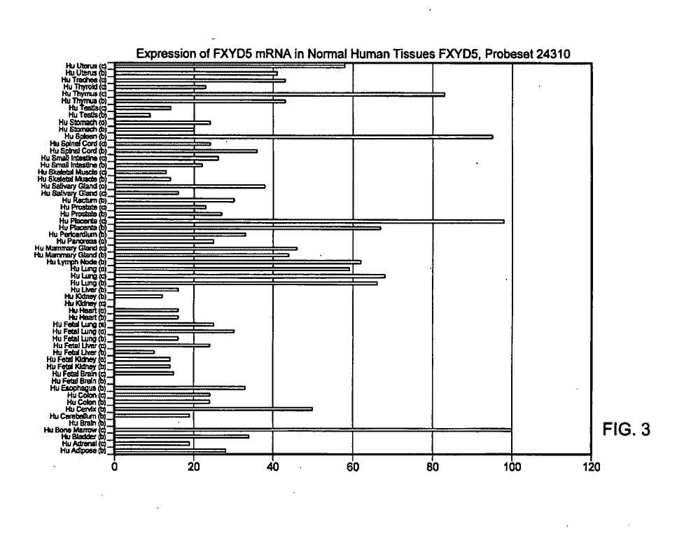

Microarray data was used to determine expression of FXYD5 in a number of primary tumors and normal tissues. The results are depicted graphically in FIGS. 1-4, which show that FXYD5 is significantly upregulated in breast and colon tumors. In one experiment, the results of which are depicted in FIG. 1, mRNA was isolated from laser capture microdissected (LCM) colon cancer, breast cancer and prostate cancer tissues, and the mRNA was compared to either a pool of respective normal tissue (RSM=reference standard mix) or normal cells adjacent to the cancer cells within each tissue sample. Samples within the A section of the x-axis are from primary breast cancer, LCM; samples within the B section are normal breast, RSM; samples within the C section are metastatic colon cancer, LCM; samples within the D section are normal colon, LCM; samples within the E section are primary colon cancer; samples within the F section are normal colon, RSM; samples within the G section are normal prostate, LCM; samples within the H section are primary prostate cancer, LCM; and samples within the I section are normal prostate, RSM. Samples were tested by oligonucleotide array analysis on Affyinetrix® GeneChips® (Affymetrix, Inc., Santa Clara, Calif.). Expression of FXYD5 mRNA in normal human tissues is shown in FIGS. 2 and 3, using FXYD5 probesets 17989 and 24320, respectively.

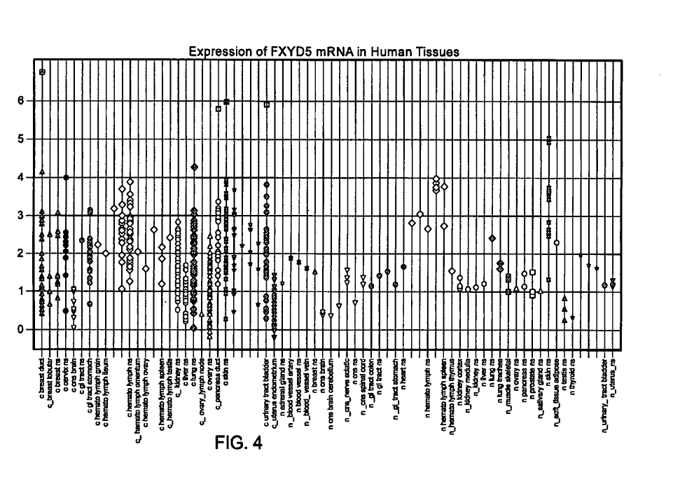

A graphical representation of an oligonucleotide array analysis of FXYD5 mRNA expression in cancerous and normal tissues using a Human Genome U133 Plus 2.0 Array (Affymetrix, Inc.) is shown in FIG. 4. Normal and cancerous tissue types are presented along the horizontal axis. Cancerous tissues are labeled with a ‘c_’ (e.g., “c_breast duct,” which represents a breast cancer tissue sample), and normal tissues are labeled with an ‘n_’. The tissue types are further labeled with respect to the type and subtype of the tissue, if known. For example, “c_breast duct” is a cancerous tissue from a breast cancer that was localized in a breast duct. If the subtype was not clear during surgical removal or was unknown, the label includes, ‘ns’ for ‘non-specified.’ Each spot on the vertical axis of FIG. 4 represents a tissue sample from a single patient, and the height of each spot on the vertical axes (linear) represents the relative expression level of the probeset Before performing an analysis, each probeset was calibrated by analyzing the behavior of its constituent probes across a large, diverse set of samples. This calibration measured the relative sensitivity of each probe, and the range of intensities within which the probeset response was linear between probes. Intensities below this range are called “undetected” while those above it are called “saturated.” Because of variation in the hybridization and labeling efficiency between samples, each array was normalized after applying the calibrations. This caused the upper and lower limits of the range, in terms of gene expression, to vary somewhat from sample to sample.

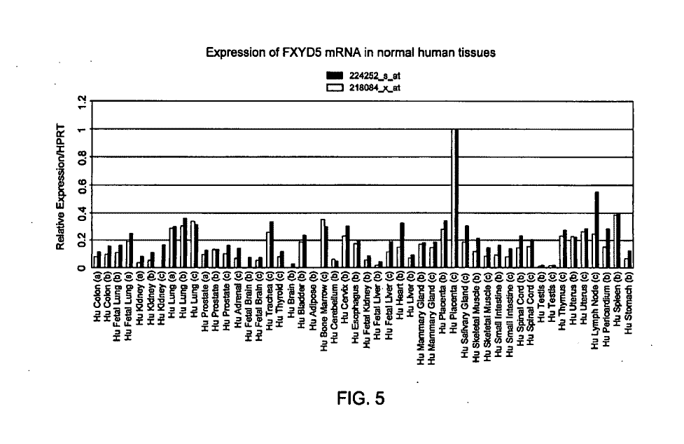

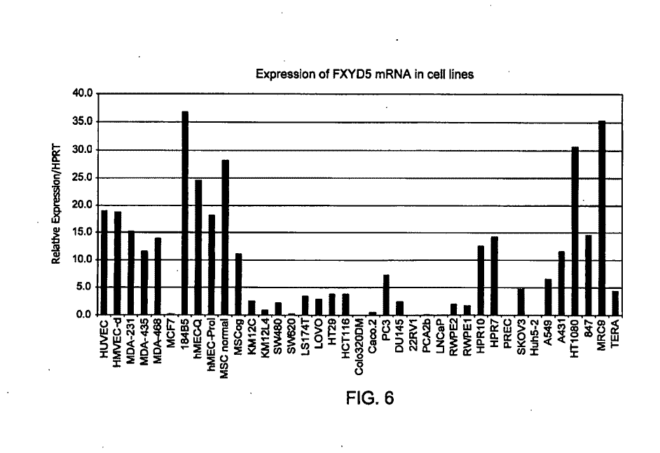

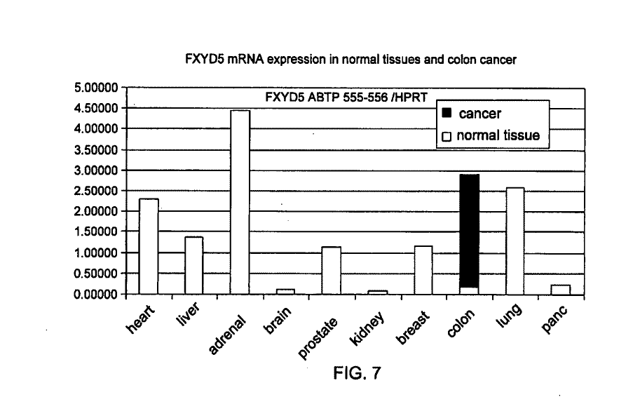

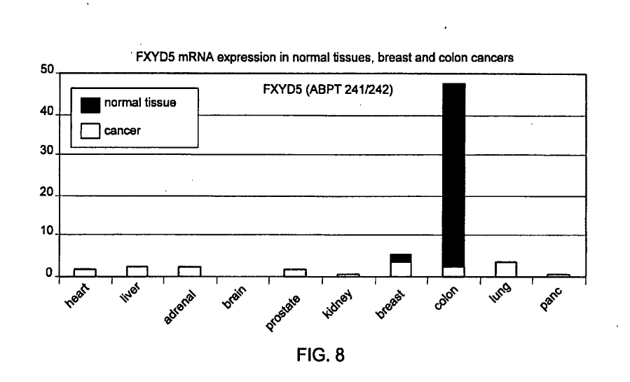

Reverse-transcription-coupled polymerase chain reaction (RT-PCR) was also used to examine relative FXYD5 mRNA levels in normal and cancer tissue samples (FIGS. 5-8). Various normal or cancer tissues or cell lines were compared by semi-quantitative RT-PCR (GeneAmp®, Applied Biosystems, Foster City, Calif.) using different primer sets. Data from the primers 224252_s_at and 218084_x_at are depicted in FIG. 5, which shows FXYD5 expression was upregulated in placental tissue as compared to other normal tissues tested. Data depicted in FIG. 6 show that FXYD5 expression was highest in HUVEC, HMVEC-d, MDA-231, 184B5, hMEC-Q, hMEC-Prol, MSC-normal, HT1090, and MRC9 cell lines. Data from the primer set named “ABTP 241-242” are depicted in FIG. 8, which shows that FXYD5 expression was highest in mda231, A431, skov3, HMEC, PREC, and MRC9 cell lines. Data from the primer set named “ABTP 555-556” are depicted in FIG. 7, which shows high levels of FXYD5 expression in colon cancer tissue. Low/no FXYD5 expression was observed in normal colon tissue.

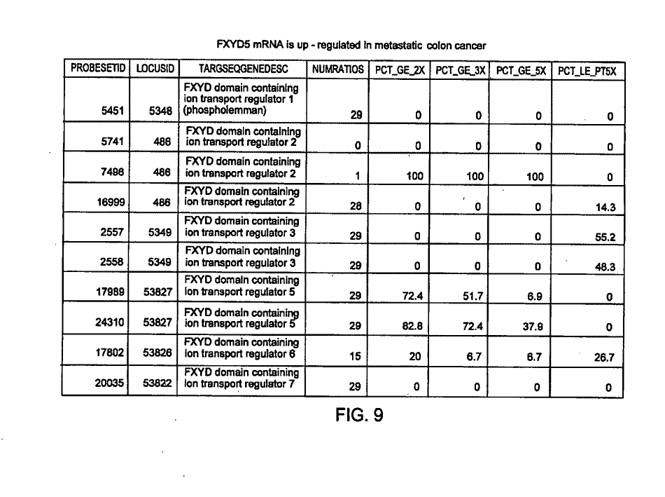

Expression of mRNA of various FXYD (FXYD 1, FXYD2, FXYD3, FXYD5, FXYD6, and FXYD7) family members was examined in colon cancer samples. As shown in FIG. 9, FXYD2 and FXYD5 expression was upregulated in colon cancer cells. Expression of the other FXYD family members was low or absent.

### Example 2

### FXYD5 Protein Expression Analyses

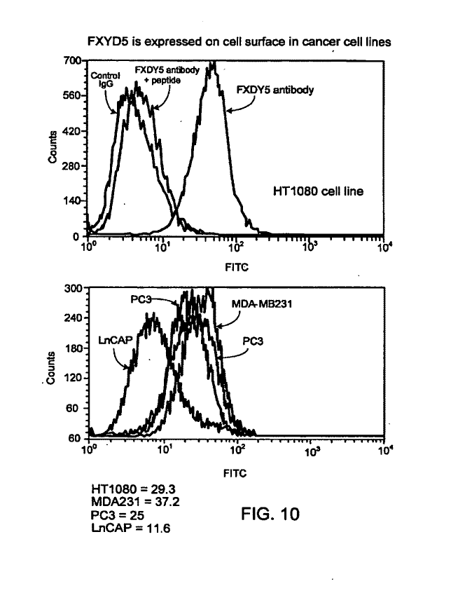

FACS. Flow cytometric (FACS) analysis was used to determine cell-surface expression of FXYD5 on various cancer cell lines. FACS analysis of non-permeabilized HT1080, MDA231, PC3, and LnCAP cells stained with an anti-FXYD5 antibody revealed that all of these cell lines expressed FXYD5 on the cell surface (FIG. 10, lower panel). Mean numbers next to the lower panel indicate the relative position of each cell line. MDA231 cells exhibited the highest level of cell-surface FXYD5 expression, followed by HT1080 cells, PC3, and LnCAP cells. The specificity of staining was confirmed with peptide competition (FIG. 10, upper panel).

Immunoldstochemistry. Immunohistochemistry (IHC) was performed on human tissues using an anti-FXYD5 antibody. IHC revealed a lack of FXYD5 expression in normal colon. Colon cancer, liver metastatic and prostate cancer tissues were positive for FXYD5 protein expression (data not shown).

### Example 3

### FXYDS siRNA and Antisense Oligonucleotides Inhibit Soft Agar Growth

PC3 and HT29 cells were transfected with siRNA or antisense oligonucleotides to determine the effect of FXYD5 inhibition on anchorage-independent growth.

For the siRNA experiments, PC3 cells were transfected with one of the following: an siRNA against FXYD5, C295-4si (CCAGATGCAGTCTACACAGAA; SEQ ID NO:23) siRNA Eg5si as a positive control; or PGL3si as a negative control. PGL3si targets unrelated gene sequences. A fourth set of cells was untransfected.

For the antisense experiments, PC3 or HT29 cells were transfected with one of the following antisense oligonucleotides: C109-3, which targets Eg5, as a control; C295-3, which targets FXYD5; or C295-4, which also targets FXYD5. Cells were also transfected with oligonucleotides containing the reverse complement of each sequence, as a negative control. The cells were plated in 0.35% soft agar and growth quantitated using Alamar Blue after 7 days in culture.

The effect of FXYD5 gene expression upon anchorage-independent cell growth of the cells was measured by colony formation in soft agar. Soft agar assays were performed by first coating a non-tissue culture treated plate with Poly-HEMA to prevent cells from attaching to the plate. Non-transfected cells were harvested using trypsin and washing twice in media. The cells were counted using a hemacytometer and resuspended to le cells per ml in media. Fifty μl aliquots were placed in polyHEMA coated 96-well plates and transfected. For each transfection mixture, a carrier molecule, preferably a lipitoid or cholesteroid, was prepared to a working concentration of 0.5 nM in water, sonicated to yield a uniform solution, and filtered through a 0.45 μm PVDF membrane.

The antisense, siRNA, or control oligonucleotide was then prepared to a working concentration of 100 μM in sterile Millipore water. The oligonucleotides were further diluted in OptiMEM™ (Gibco/BRL) in a microfuge tube to 2 μM, or approximately 20 μg oligo/ml of OptiMEM™. In a separate microfuge tube, lipitoid or cholesteroid, typically in the amount of about 1.5-2 mmol lipitoid/μg oligonucleotide, was diluted in the same volume of OptiMEM™ used to dilute the oligonucleotide. The diluted oligonucleotide was immediately added to the diluted lipitoid and mixed by pipetting up and down. Antisense oligonucleotide was added to the cells to a final concentration of about 300 nM. siRNAs were added to a final concentration of about 66 nM. Following transfection at 37° C. for about 30 minutes, 3% GTG agarose was added to the cells for a final concentration of 0.35% agarose by pipetting up and down. After the cell layer agarose solidified, 100 μl of media was dribbled on top of each well. Colonies formed in about 7 days. For a read-out of growth, 20 μl of Alamar Blue was added to each well and the plate was shaken for about 15 minutes. Fluorescence readings (530 nm excitation/590 nm emission) were taken after incubation for 6-24 hours.

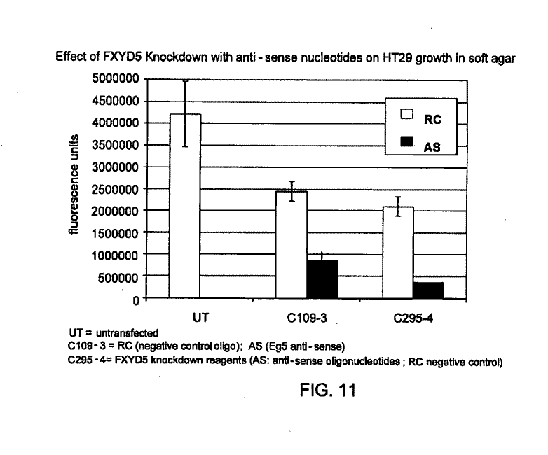

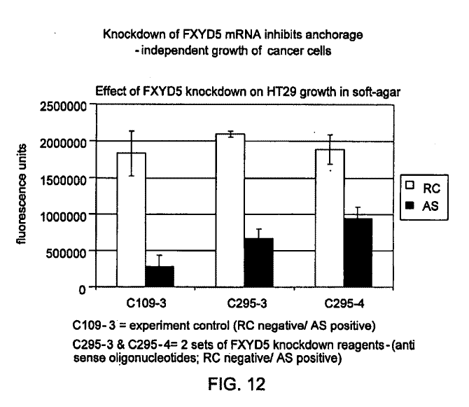

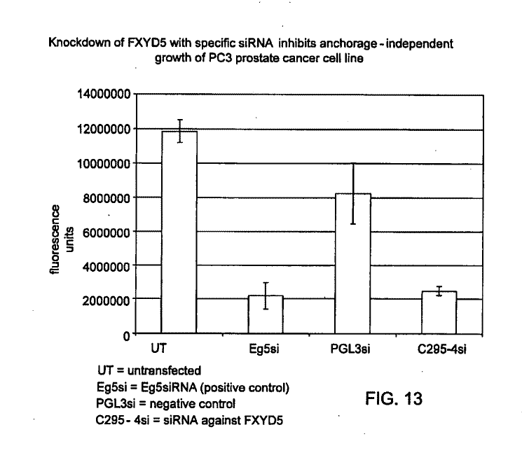

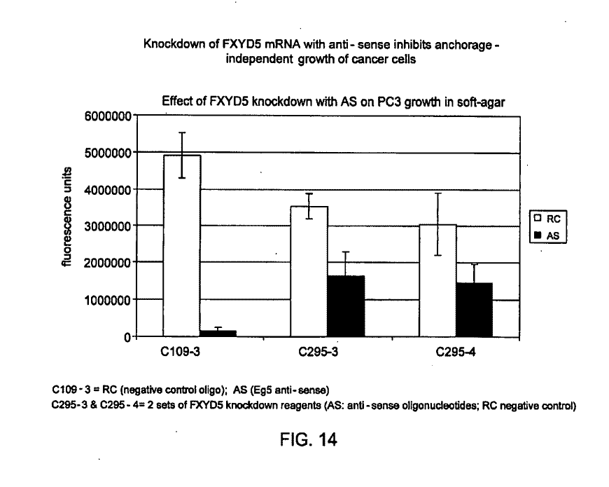

The results of the antisense experiments in PC3 cells are depicted in FIG. 14; results of the siRNA experiments on PC3 cells are depicted in FIG. 13. FXYD5 siRNAs inhibited anchorage independent growth of cancer cells to levels comparable to the positive control. The results of the antisense experiments on HT29 cells are depicted in FIGS. 11 and 12. Antisense oligonucleotides targeting FXYD5 inhibited anchorage-independent growth of cancer cells.

Inhibition of colony formation in cancer cell lines using FXYD5 modulators indicates that FXYD5 plays a role in production and/or maintenance of the metastatic phenotype.

### Example 4

### FXYD5 siRNA Induces Cytotoxicity in Cancer Cells but not Normal Cells

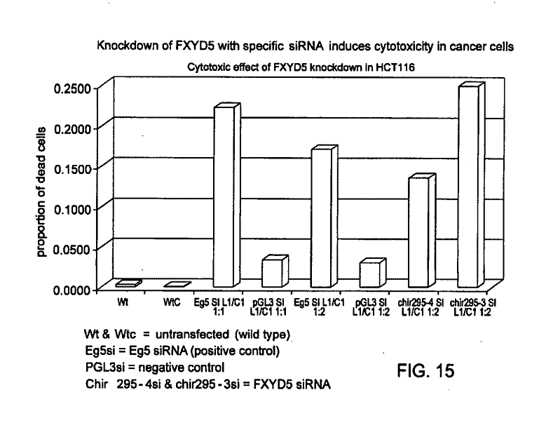

Experiments with FXYD5 siRNA revealed that inhibition of FXYD5 is cytotoxic to cancer cells but not normal cells. Briefly, HCT116, PC3, or normal non-tumorigenic fibroblast (MRC9) cells were transfected with one of the following siRNAs: Eg5si (positive control), PGL3si (negative control), C295-3 (FXYD5 siRNA), or C295-4 (FXYD5 siRNA). Cytotoxicity of untransfected cells was also determined. Transfections were performed as described in the previous example. For FIGS. 15-17, cytotoxicity was monitored by measuring the amount of LDH enzyme released in the medium due to membrane damage. The activity of LDH was measured using the Cytotoxicity Detection Kit from Roche Molecular Biochemicals. The data is provided as a ratio of LDH released in the medium vs. the total LDH present in the well at the same time point and treatment (rLDHALDH).

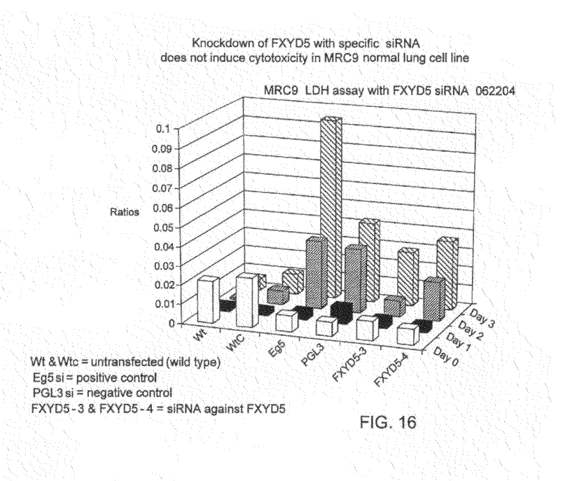

As depicted in FIGS. 15-16. FXYD5 siRNAs induced cytotoxicity in the HCT116 cells and PC3 cells to a greater extent than negative control siRNA (FIGS. 15 and 16, respectively). By day three, the extent of cytotoxicity in MRC9 cells induced by FXYD5 siRNA was comparable to that of the negative control siRNA (FIG. 16). Thus, cancer cells exhibit a heightened sensitivity to FXYD5 inhibition relative to non-cancerous cells. Agents which antagonize FXYD5 can exhibit therapeutic indices which are suitable for treatment of neoplastic disorders.

### Example 5

### FXYD5 Inhibition in Combination with Chemotherapy

FXYD5 inhibition was examined in conjunction with chemotherapeutic treatment of cells. Oligonucleotide transfections were performed as described in Example 3 LnCaP cells were transfected with one of the following antisense oligonucleotides: CHIR295-3, which targets FXYD5; CHIR295-4, which targets FXYD5; an antisense oligonucleotide which targets Bc12; or a reverse complement of one of these. Untransfected cells were also analyzed. Subsets of cells were also treated with methotrexate (MTX) or doxorubicin (Doxo), at various concentrations. Cytotoxicity was determined by measuring the amount of LDH enzyme released in the medium due to membrane damage, as described in the previous example.

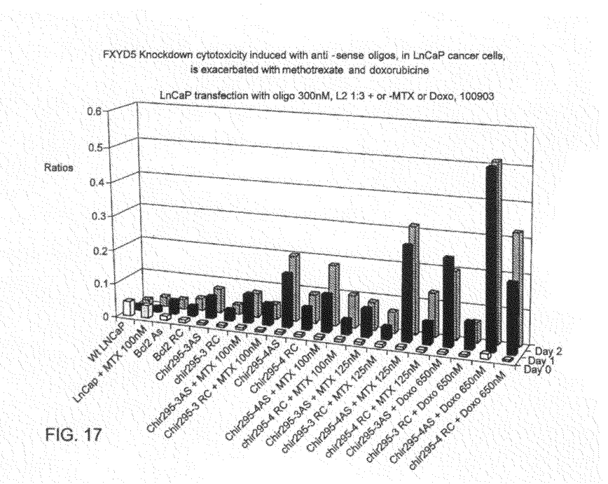

As shown in FIG. 17, combination treatment with a chemotherapeutic agent had an additive effect on cell cytotoxicity.

### Example 6

### FXYD5 Epitopes

Linear epitopes of FXYD5 for antibody recognition and preparation can be identified by any of numerous methods known in the art. Some example methods include probing antibody-binding ability of peptides derived from the amino acid sequence of the antigen. Binding can be assessed by using BIACORE or FLISA methods. Other techniques include exposing peptide libraries on planar solid support (“chip”) to antibodies and detecting binding through any of multiple methods used in solid-phase screening. Additionally, phage display can be used to screen a library of peptides with selection of epitopes after several rounds of biopanning.

### Example 7

### Modulators

In some embodiments, FXYD5 modulators are selected from the group consisting of:

(a) an antibody that binds an epitope in the extracellular domain (ECD) of FXYD5;

(b) an isolated double-stranded RNA (dsRNA) comprising a first strand of nucleotides comprising at least 19 consecutive nucleotides of a sequence set forth in SEQ ID NOs:1, 8, 9, and 12-26, or a full complement thereof; and a second strand of nucleotides comprising a sequence substantially complementary to the first strand, wherein the dsRNA molecule is less than 890 nucleotides long;

(c) an isolated nucleic acid molecule comprising at least 10 consecutive nucleotides of a sequence at least 90% identical to a sequence selected from the group consisting of SEQ ID NOs:1, 5-21, 24 and 25, or a complement thereof;

(d) a small molecule;

(e) a mimetic;

(f) a soluble receptor; and

(g) a decoy.

In some embodiments, FXYD5 modulators comprise or are directed to antigenic regions of the FXYD5 polypeptide. In some embodiments of the present invention, FXYD5 modulators comprise and/or specifically bind to one or more sequences of SEQ ID NO:2.

In some embodiments the FXYD5 modulator is a monoclonal antibody that binds one or more epitopes of SEQ ID NO:2, wherein each of said epitopes consists of between about 6 and 20 contiguous amino acids of SEQ ID NO:2. In some embodiments, the antibody is a monoclonal antibody which specifically binds to an epitope of FXYD5 other than an epitope selected from the group consisting of: Thr19-Ala33 (SEQ ID NO:3), Leu54-Asp82 (SEQ ID NO:4), Pro89-Ala97 (SEQ ID NO:5), Pro100-Lys125 (SEQ ID NO:6), and Ser127-Phe135 (SEQ ID NO:7).

In some embodiments, FXYD5 modulators are isolated nucleic acid molecules comprising at least 10 consecutive nucleotides of a sequence at least 90% identical to a sequence selected from the group consisting of SEQ ID NOs:1, 8, 9, and 12-26, or a full complement thereof. In some embodiments FXYD5 modulators are antisense or siRNA oligonucleotides and have a sequence selected from the group consisting of SEQ ID NOs: 12-26.

FXYD5 Antisense and siRNA Oligos

| | Sequence | SEQ ID NO: |
|---|---|---|
| antisense | | |
| CHIR295-1 | GGTGAGAAGACACAGGCGACCAGA | 12 |
| CHIR295-2 | TTGAGTCTGCTGAAGAACTGGACGT | 13 |
| CHIR295-3 | GTGTCGGGACCTGAATGTGCATGAT | 14 |
| CHIR295-4 | GAGAGGTGGGCTGGAGTTCTGTGTA | 15 |
| CHIR295-5 | ATCCGTTCCTTCCAGTTGCTGGGT | 16 |
| CHIR295-8 | GCGTCGTGGTGTCATCAGTGGGAT | 17 |
| CHIR295-7 | GGATGATGATGCCTGTGATGAACAG | 18 |
| CHIR295-8 | TGATGGACTCACCTGCAACGATTCC | 19 |
| siRNA | | |
| CHIR295-1SI | CCCGGTTATGCCGGAATCGTT | 20 |
| CHIR295-2SI | CTCAACTATCATGGACATTCA | 21 |
| CHIR295-3SI | GAGGACAGACGTTGAAAGATA | 22 |
| CHIR295-4SI | CCAGATGCAGTCTACACAGAA | 23 |
| CHIR295-6SI | CAGGCATCATCATCCTCACCA | 24 |
| CHIR295-6SI | CAGGTGAGTCCATCAGAAACA | 25 |
| CHIR295-7SI | GAGGGAAGACACAGATGATGA | 26 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention.

What is claimed is:

1. A method of treating cancer or a cancer symptom in a patient in need thereof comprising administering to the patient a therapeutically effective amount of a composition comprising an FXYD5 modulator and one or more pharmaceutically acceptable carriers.

2. A method of modulating an FXYD5 activity in a patient in need thereof; the method comprising administering to the patient an amount of an FXYD5 modulator effective to modulate the FXYDS activity, and one or more pharmaceutically acceptable carriers.

3. A method of inhibiting growth of a cancer cell that expresses FXYD5 comprising administering to the cancer cell an amount of a composition comprising an FXYD5 modulator effective to inhibit growth of the cell by at least 20% as compared to a control, and one or more pharmaceutically acceptable carriers.

4. A method of inhibiting a cancer phenotype in a patient in need thereof comₚrising administering to the patient a therapeutically effective amount of a composition comprising an FXYD5 modulator, and one or more pharmaceutically acceptable carriers.

5. A method of modulating one or more activities in a cancer cell that expresses FXYD5 comprising administering to the cancer cell an amount of a composition comprising an FXYD5 modulator effective to modulate the one or more activities, and one or more pharmaceutically acceptable carriers.

6. A method for inhibiting the interaction of FXYD5 on a cancer cell with an FXYD5 ligand, comprising administering to the cancer cell an effective amount of an FXYD5 modulator in the presence of the FXYD5 ligand, thereby inhibiting interaction of FXYD5 on the cell with the FXYD5 ligand.

7. A method of inducing apoptosis in a cancer cell expressing FXYDS, the method comprising administering an effective amount of an FXYD5 modulator to the cancer cell with one or more pharmaceutically acceptable carriers.

8. The method of any of claims 1-7 wherein further comprising administering methotrexate or doxorubicine to the patient or cell.

9. The method of any of claims 1-7 wherein the FXYD5 modulator is selected from the group consisting of:
(a) an antibody that binds an epitope in the extracellular domain (ECD) of FXYD5;
(b) an isolated double-stranded RNA (dsRNA) comprising a first strand of nucleotides comprising at least 19 consecutive nucleotides of a sequence set forth in SEQ ID NOs: 1, 8, 9, and 12-26, or a full complement thereof, and a second strand of nucleotides comprising a sequence substantially complementary to the first strand, wherein the dsRNA molecule is less than 890 nucleotides long;
(c) an isolated nucleic acid molecule comprising at least 10 consecutive nucleotides of a sequence at least 90% identical to a sequence selected from the group consisting of SEQ ID NOs: 1, 8, 9, and 12-26, or a full complement thereof;
(d) a small molecule;
(e) a mimetic;
(f) a soluble receptor; and
(g) a decoy.

10. The method of any of claims 1-7 wherein the FXYD5 modulator inhibits growth of cancer cells that express FXYD5 by at least 25% in an in vitro assay to measure cell growth or apoptosis.

11. The method of any of claims 1-7 wherein the FXYD5 modulator inhibits FXYD5 expression by at least 50% as compared to a control.

12. The method of any of claims 1-7 wherein the FXYD5 modulator is an oligonucleotide having a sequence selected from the group consisting of SEQ ID NOs: 12-26.

13. The method of any of claims 1-7 wherein the FXYD5 modulator is a monoclonal antibody.

14. The method of claim 13 wherein the monoclonal antibody binds one or more epitopes of SEQ ID NO:2, wherein each of said one or more epitopes consists of between about 6 and 20 contiguous amino acids of SEQ ID NO:2.

15. The method of any of claim 1, 3 or 4 wherein the cancer is selected from the group consisting of colon cancer, breast cancer, prostatic cancer, ovarian cancer, skin cancer, esophageal cancer, liver cancer, pancreatic cancer, uterine cancer, cervical cancer, lung cancer, bladder cancer, multiple myeloma and melanoma.

16. The method of claim 15, wherein the cancer is colon adenocarcinoma or squamous cell carcinoma.

17. The method of claim 15 wherein the cancer is a breast cancer selected from the group consisting of ductal adenocarcinoma, lobular adenocarcinoma, and metastatic adenocarcinoma.

18. The method of any of claim 3, 5, 6 or 7 wherein the cancer cells are selected from the group consisting of breast cancer cells, skin cancer cells, esophageal cancer cells, liver cancer cells, pancreatic cancer cells, prostatic cancer cells, uterine cancer cells, cervical cancer cells, lung cancer cells, bladder cancer cells, ovarian cancer cells, multiple myeloma cells and melanoma cells.

19. The method of any of claim 1, 2 or 4 further comprising treating the patient with one or more of chemotherapy, radiation therapy or surgery.

20. The method of claim 1 wherein the cancer symptom is selected from the group consisting of breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis, pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreas metastases, and difficulty swallowing.

21. A method of identifying a patient susceptible to FXYD5 therapy comprising:
(a) detecting the presence or absence of FXYD5 differential expression in a patient sample, wherein the presence of FXYDS differential expression in said sample is indicative of a patient who is a candidate for FXYD5 therapy and the absence FXYDS differential expression in said sample is indicative of a patient who is not a candidate for FXYD5 therapy,
(b) administering a therapeutically effective amount of the composition of claim 1 to the patient if the patient is a candidate for FXYD5 therapy; and
(c) administering a conventional cancer therapeutic to the patient if the patient is not a candidate for FXYD5 therapy.

22. A method for detecting one or more cancer cells expressing FXYD5 in a sample comprising contacting the sample with a composition comprising a FXYD5 modulator linked to an imaging agent and detecting the localization of the imaging agent in the sample.

23. The method of claim 22 wherein the composition comprises an FXYD5 antibody conjugated to an imaging agent.

24. The method of claim 22 wherein the imaging agent is ¹⁸F, ⁴³K, ⁵²Fe, ⁵⁷ Co, ⁶⁷CU, ⁶⁷Ga, ⁷⁷Br, ⁸⁷MSr, ⁸⁶Y, ⁹⁰Y, ⁹⁹MTc, ¹¹¹In, ¹²³I, ¹²⁵I, ¹²⁷Cs, ¹²⁹Cs, ¹³¹I, ¹³²I, ¹⁹⁷Hg, ²⁰³Pb, or ²⁰⁶Bi.

25. A method of expressing an anti-FXYD5 antibody in a CHO or myeloma cell wherein the anti-FXYD5 antibody inhibits one or more FXYD5-related biological activities, the method comprising expressing a nucleic acid encoding the anti-FXYD5 antibody in said CHO or myeloma cell.

26. A method of identifying a cancer inhibitor, said cancer characterized by overexpression of FXYD5 compared to a control, said method comprising contacting a cell expressing FXYD5 with a candidate compound and a FXYD5 ligand, and determining whether a downstream marker of FXYD5 is modulated, wherein modulation of the downstream marker is indicative of a cancer inhibitor.

27. The method of claim 26 wherein the downstream marker is E-cadherin.

28. A method for determining the susceptibility of a patient to a FXYD5 modulator comprising detecting evidence of differential expression of FXYD5 in said patient's cancer sample, wherein evidence of differential expression of FXYD5 is indicative of the patient's susceptibility to said FXYD5 modulator.

29. A composition comprising a FXYD5 modulator and one or more pharmaceutically acceptable carriers, wherein the FXYD5 modulator comprises.
(a) an antibody that binds an epitope in the extracellular domain (ECD) of FXYD5;
(b) an isolated double-stranded RNA (dsRNA) comprising a first strand of nucleotides comprising at least 19 consecutive nucleotides of a sequence set forth in SEQ ID NOs: 1, 8, 9, and 12-26, or a full complement thereof, and a second strand .of nucleotides comprising a sequence substantially complementary to the first strand, wherein the dsRNA molecule is less than 890 nucleotides long;
(c) an isolated nucleic acid molecule comprising at least 10 consecutive nucleotides of a sequence at least 90% identical to a sequence selected from the group consisting of SEQ ID NOs: I, 8, 9, and 12-26, or a full complement thereof;
(d) a small molecule;
(e) a mimetic;
(f) a soluble receptor; and
(g) a decoy.

30. The composition of claim 29 further comprising a chemotherapeutic agent.

31. The composition of claim 30 wherein the chemotherapeutic is methotrexate or doxorubicins.

32. The composition of claim 29 wherein the FXYD5 modulator inhibits one or more of cancer cell growth, cancer cell survival, tumor formation, and cancer cell proliferation by at least 50% compared to a control.

33. The composition of claim 29 wherein the composition is a sterile injectable.

34. The composition of claim 29 wherein the isolated nucleic acid molecule is a dsRNA, a short interfering RNA (siRNA), or an antisense oligonucleotide.

35. The composition of claim 29 wherein the FXYD5 modulator is a monoclonal antibody which specifically binds to a FXYD5 polypeptide with an affinity of at least 1×10⁸Ka.

36. The composition of claim 35, wherein the FXYD5 polypeptide has a sequence at least 95% identical to SEQ ID NO:2.

37. The composition of claim 35 wherein the FXYD5 polypeptide has a sequence of SEQ ID NO:2.

38. The composition of claim 35, wherein the FXYD5 polypeptide is encoded by a nucleic acid comprising a sequence at least 95% identical to a sequence selected from SEQ ID NO:1 and SEQ ID NO:9.

39. The composition of claim 35 wherein the monoclonal antibody is a chimeric antibody, a human antibody, a humanized antibody, a single-chain antibody, a bi-specific antibody, a multi-specific antibody, or a Fab fragment.

40. The composition of claim 35 wherein the monoclonal antibody binds to one or more epitopes of SEQ ID NO:2.

41. The composition of claim 35 wherein the monoclonal antibody specifically binds to one or more epitopes in amino acids 22-145 of SEQ ID NO:2.

42. An isolated cell that produces the antibody of claim 35.

43. A hybridoma that produces the antibody of claim 35.

44. A non-human transgenic animal that produces an antibody of claim 35.

45. An isolated epitope-bearing polypeptide comprising one or more epitopes of SEQ ID NO:2.

46. A polynucleotide that encodes an isolated epitope-bearing polypeptide of claim 45.

47. The epitope-bearing polypeptide of claim 45, wherein each of said one or more epitopes consists of between about 6 and about 20 contiguous amino acids of SEQ ID NO:2.

48. The epitope-bearing polypeptide of claim 45, wherein each of said one or more epitopes consist of between 10 and about 20 contiguous amino acids of SEQ ID NO:2.

49. The epitope-bearing polypeptide of claim 45, wherein at least one of said one or more cpitopes consists of at least 21 contiguous amino acids of SEQ ID NO:2.

50. The epitope-bearing polypeptide of claim 45, which comprises at least two epitopes of SEQ ID NO:2 and wherein each of said epitopes consists of between about 6 and 20 contiguous amino acids of SEQ ID NO:2.

51. An isolated FXYD5 antibody which is obtained by immunization of a subject with the epitope-bearing polypeptide of claim 45.
